(19) United States
(12) Patent Application Publication
(10) Pub. No.: US 2022/0059187 A1
(43) Pub. Date: Feb. 24, 2022

(54) METHODS OF DETECTING NUCLEIC ACID BARCODES

(71) Applicant: Oxford Nanopore Technologies Limited, Oxford (GB)

(72) Inventors: Stuart William Reid, Oxford (GB); Eoghan Donal Harrington, New York, NY (US)

(73) Assignee: Oxford Nanopore Technologies Limited, Oxford (GB)

(21) Appl. No.: 17/395,895

(22) Filed: Aug. 6, 2021

Related U.S. Application Data
(60) Provisional application No. 63/063,178, filed on Aug. 7, 2020.

(51) Int. Cl. G16B 30/10; C12Q 1/6876; C12Q 1/70

(52) U.S. Cl. CPC G16B 30/10; C12Q 2600/16; C12Q 1/70; C12Q 1/6876

(57) ABSTRACT

Provided herein, in some embodiments, are methods of determining whether a target nucleic acid comprises a particular barcode sequence.

### RELATED APPLICATION

This application claims the benefit under 35 U.S.C. § 119(e) of U.S. Provisional Application U.S. Ser. No. 63/063,178 filed Aug. 7, 2020, the contents of which are incorporated herein by reference.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Aug. 4, 2020 is named 0036670103US01-SEQ-MSB and is 3,760 bytes in size.

### BACKGROUND

Nucleic acid sequencing can be used to evaluate biological samples for one or more indicia of disease. For example, nucleic acid sequencing can be used to determine whether a patient sample contains one more genomic mutations associated with a disease or disorder, or to interrogate a patient sample for the presence of one or more sequences indicative of an infection (e.g., a viral, bacterial, or other microbial infection).

In order to process many samples efficiently, nucleic acid sequencing is often performed in multiplexed sequencing reactions that allow nucleic acid templates obtained from many different samples (e.g., from different patients) to be sequenced together in the same reaction. In a typical multiplexed reaction, nucleic acids from different samples are tagged by attaching a sample-specific barcode to the nucleic acids prior to combining them for sequencing. The resulting sequencing data contains many different sequences having different barcodes. An initial step in the sequence analysis can involve identifying the barcodes associated with the different sequences in order to match the sequences to the samples they were obtained from. Barcode misidentification can be a source of error that leads to incorrect or inconclusive diagnosis or disease detection. Accordingly, new methods of identifying nucleic acids having a particular barcode are needed.

### SUMMARY

Methods and systems of the application are useful to identify nucleic acid barcode sequences in data obtained from multiplexed sequencing reactions. The sequencing data can be obtained from any sequencing platform, for example using any sequencing protocol that involves adding barcodes to different nucleic acids (e.g., from different samples) and combining the barcoded nucleic acids in a common sequencing reaction. The inventors have discovered a reliable and robust method of detecting barcodes that involves generating an alignment between a target nucleic acid and a reference nucleic acid prior to scoring the aligned target nucleic acid against a scoring region of the reference nucleic acid that includes, in some embodiments, a particular barcode sequence and flanking nucleotides from fixed context sequences (e.g., primer sequences). Accordingly, in some aspects, the disclosure provides a method of determining whether a target nucleic acid (e.g., a target nucleic acid in a multiplexed sample) comprises a particular barcode sequence.

In some aspects, the disclosure provides a method comprising using at least one computer hardware processor to perform:
(i) generating an alignment between at least a segment of a target nucleic acid and at least a segment of a reference nucleic acid, wherein the reference nucleic acid comprises a barcode sequence and a first context sequence,
(ii) determining a sequence similarity between a scoring region of the reference nucleic acid and a corresponding segment of the target nucleic acid, wherein the corresponding segment is identified based on the alignment,
wherein the scoring region comprises at least a portion of the barcode sequence and at least one and no more than a first threshold number of nucleotides of a first context sequence; and
(iii) determining whether the target nucleic acid comprises the barcode sequence based on the sequence similarity between the target nucleic acid and the scoring region of the reference nucleic acid.

Further aspects of the disclosure provide systems for performing any of the methods described herein. For example, in some embodiments, a system comprises at least one computer hardware processor; and at least one non-transitory computer readable storage medium storing processor executable instructions that, when executed by the at least one computer hardware processor, cause the at least one computer hardware processor to perform:
(i) generating an alignment between at least a segment of a target nucleic acid and at least a segment of a reference nucleic acid, wherein the reference nucleic acid comprises a barcode sequence and a first context sequence;
(ii) determining a sequence similarity between a scoring region of the reference nucleic acid and a corresponding segment of the target nucleic acid, wherein the corresponding segment is identified based on the alignment,
wherein the scoring region comprises at least a portion of the barcode sequence and at least one and no more than a first threshold number of nucleotides of a first context sequence; and
(iii) determining whether the target nucleic acid comprises the barcode sequence based on the sequence similarity between the target nucleic acid and the scoring region of the reference nucleic acid.

Still further aspects of the disclosure provide at least one non-transitory computer readable storage medium storing processor executable instructions for performing any of the methods described herein. For example, in some embodiments, at least one non-transitory computer readable storage medium storing processor executable instructions that, when executed by at least one computer hardware processor, causes the at least one computer hardware processor to perform:
(i) generating an alignment between at least a segment of a target nucleic acid and at least a segment of a reference nucleic acid, wherein the reference nucleic acid comprises a barcode sequence, a first context sequence, and a second context sequence,
(ii) determining a sequence similarity between a scoring region of the reference nucleic acid and a corresponding segment of the target nucleic acid, wherein the corresponding segment is identified based on the alignment,
   wherein the scoring region comprises at least a portion of the barcode sequence, at least one and no more than a first threshold number of nucleotides of a first context sequence and no more than a second threshold number of nucleotides of a second context sequence; and
(iii) determining whether the target nucleic acid comprises the barcode sequence based on the sequence similarity between the target nucleic acid and the scoring region of the reference nucleic acid.

In some embodiments, the reference nucleic acid further comprises a second context sequence and the scoring region further comprises no more than a second threshold of nucleotides of the second context sequence. In some embodiments, prior to generating the alignment in step (i), generating an initial alignment between the at least a segment of the target nucleic acid and an initial region of the reference nucleic acid that contains at least the barcode sequence and the first context sequence, wherein generating the alignment in step (i) is performed based on the initial alignment, and wherein the segment of the reference nucleic acid is the scoring region of the reference nucleic acid.

In some aspects, the disclosure provides a method comprising using at least one computer hardware processor to perform:
(i) generating a plurality of alignments between at least a segment of a target nucleic acid and at least a segment of each of a plurality of reference nucleic acids, wherein each of the plurality of reference nucleic acids comprises a respective barcode sequence and a first context sequence;
(ii) determining a respective plurality of sequence similarities between scoring regions of the plurality of reference nucleic acids and the target nucleic acid, wherein the plurality of sequence similarities comprises a first sequence similarity, the plurality of reference nucleic acids comprises a first reference nucleic acid having a first scoring region, the plurality of respective barcode sequences comprises a first barcode sequence, and the plurality of alignments comprise a first alignment between the at least a segment of the target nucleic acid and at least a segment of the first reference nucleic acid, the determining comprising:
   determining the first sequence similarity between the first scoring region of the first reference nucleic acid and a corresponding segment of the target nucleic acid, wherein the corresponding segment is identified based on the first alignment, and the first scoring region comprises at least a portion of the first barcode sequence and at least one and no more than a first threshold number of nucleotides of the first context sequence; and
(iii) identifying which of the plurality of respective barcode sequences is contained in the target nucleic acid based on the plurality of sequence similarities.

In some aspects, a system comprises at least one computer hardware processor; and at least one non-transitory computer readable storage medium storing processor executable instructions that, when executed by the at least one computer hardware processor, cause the at least one computer hardware processor to perform:
(i) generating a plurality of alignments between at least a segment of a target nucleic acid and at least a segment of each of a plurality of reference nucleic acids, wherein each of the plurality of reference nucleic acids comprises a respective barcode sequence, a first context sequence, and a second context sequence;
(ii) determining a respective plurality of sequence similarities between scoring regions of the plurality of reference nucleic acids and the target nucleic acid, wherein the plurality of sequence similarities comprises a first sequence similarity, the plurality of reference nucleic acids comprises a first reference nucleic acid having a first scoring region, the plurality of respective barcode sequences comprises a first barcode sequence, the plurality of alignments comprise a first alignment between the at least a segment of the target nucleic acid and at least a segment of the first reference nucleic acid, the determining comprising:
   determining the first sequence similarity between the first scoring region of the reference nucleic acid a corresponding segment of the target nucleic acid, wherein the corresponding segment is identified based on the first alignment, wherein the first scoring region comprises at least a portion of the first barcode sequence, at least one and no more than a first threshold number of nucleotides of the first context sequence and no more than a second threshold number of nucleotides of the second context sequence; and
(iii) identifying which of the plurality of respective barcode sequences is contained in the target nucleic acid based on the plurality of sequence similarities.

In some aspects, at least one non-transitory computer readable storage medium storing processor executable instructions that, when executed by at least one computer hardware processor, causes the at least one computer hardware processor to perform:
(i) generating a plurality of alignments between at least a segment of a target nucleic acid and at least a segment of each of a plurality of reference nucleic acids, wherein each of the plurality of reference nucleic acids comprises a respective barcode sequence, a first context sequence, and a second context sequence;
(ii) determining a respective plurality of sequence similarities between scoring regions of the plurality of reference nucleic acids and the target nucleic acid, wherein the plurality of sequence similarities comprises a first sequence similarity, the plurality of reference nucleic acids comprises a first reference nucleic acid having a first scoring region, the plurality of respective barcode sequences comprises a first barcode sequence, the plurality of alignments comprise a first alignment between the at least a segment of the target nucleic acid and at least a segment of the first reference nucleic acid, the determining comprising:
   determining the first sequence similarity between the first scoring region of the reference nucleic acid a corresponding segment of the target nucleic acid, wherein the corresponding segment is identified based on the first alignment, wherein the first scoring region comprises at least a portion of the first barcode sequence, at least one and no more than a first threshold number of nucleotides of the first context sequence and no more than a second threshold number of nucleotides of the second context sequence; and
(iii) identifying which of the plurality of respective barcode sequences is contained in the target nucleic acid based on the plurality of sequence similarities.

In some embodiments, each of the plurality of reference nucleic acids further comprises a second context sequence and the first scoring region further comprises no more than a second threshold of nucleotides of the second context sequence. In some embodiments, prior to generating the plurality of alignments in step (i), generating a plurality of initial alignments between the at least a segment of the target nucleic acid and an initial region of each of the reference nucleic acids that contains at least the barcode sequence and the first context sequence, wherein generating the plurality of alignments in step (i) is performed based on the plurality of initial alignments, and wherein the segment of the first reference nucleic acid is the first scoring region of the reference nucleic acid. In some embodiments, each of the plurality of reference nucleic acids comprises a respective barcode sequence having a different and unique nucleotide sequence. In some embodiments, the plurality of reference nucleic acids comprises at least 8, 16, 32, 64, 96, 192, 288, 384, or 480 reference nucleic acids.

Some aspects of the disclosure provide a method comprising using at least one computer hardware processor to perform:
(i) generating a plurality of alignments between at least a segment of each of a plurality of target nucleic acids and at least a segment of a reference nucleic acid, wherein the reference nucleic acid comprises a barcode sequence and a first context sequence;
(ii) determining a respective plurality of sequence similarities between a scoring region of the reference nucleic acid and the plurality of target nucleic acids, wherein the plurality of sequence similarities comprises a first sequence similarity, and wherein the plurality of alignments comprise a first alignment between the at least a segment of the first target nucleic acid and the reference nucleic acid, the determining comprising:
   determining the first sequence similarity between the scoring region of the reference nucleic acid and a corresponding segment of the first target nucleic acid, wherein the corresponding segment is identified based on the first alignment, wherein the scoring region comprises at least a portion of the barcode sequence and at least one and no more than a first threshold number of nucleotides of the first context sequence; and
(iii) identifying which of the plurality of target nucleic acids contains the barcode sequence based on the plurality of sequence similarities.

In some aspects, a system comprises at least one computer hardware processor; and at least one non-transitory computer readable storage medium storing processor executable instructions that, when executed by the at least one computer hardware processor, cause the at least one computer hardware processor to perform:
(i) generating a plurality of alignments between at least a segment of each of a plurality of target nucleic acids and at least a segment of a reference nucleic acid, wherein the reference nucleic acid comprises a barcode sequence, a first context sequence, and a second context sequence;
(ii) determining a respective plurality of sequence similarities between a scoring region of the reference nucleic acid and the plurality of target nucleic acids, wherein the plurality of sequence similarities comprises a first sequence similarity, and wherein the plurality of alignments comprise a first alignment between the at least a segment of the first target nucleic acid and the reference nucleic acid, the determining comprising:
   determining the first sequence similarity between the scoring region of the reference nucleic acid and a corresponding segment of the first target nucleic acid, wherein the corresponding segment is identified based on the first alignment, wherein the scoring region comprises at least a portion of the barcode sequence, at least one and no more than a first threshold number of nucleotides of the first context sequence and no more than a second threshold number of nucleotides of the second context sequence; and
(iii) identifying which of the plurality of target nucleic acids contains the barcode sequence based on the plurality of sequence similarities.

In some aspects, at least one non-transitory computer readable storage medium storing processor executable instructions that, when executed by at least one computer hardware processor, causes the at least one computer hardware processor to perform:
(i) generating a plurality of alignments between at least a segment of each of a plurality of target nucleic acids and at least a segment of a reference nucleic acid, wherein the reference nucleic acid comprises a barcode sequence, a first context sequence, and a second context sequence;
(ii) determining a respective plurality of sequence similarities between a scoring region of the reference nucleic acid and the plurality of target nucleic acids, wherein the plurality of sequence similarities comprises a first sequence similarity, and wherein the plurality of alignments comprise a first alignment between the at least a segment of the first target nucleic acid and the reference nucleic acid, the determining comprising:
   determining the first sequence similarity between the scoring region of the reference nucleic acid and a corresponding segment of the first target nucleic acid, wherein the corresponding segment is identified based on the first alignment, wherein the scoring region comprises at least a portion of the barcode sequence, at least one and no more than a first threshold number of nucleotides of the first context sequence and no more than a second threshold number of nucleotides of the second context sequence; and
(iii) identifying which of the plurality of target nucleic acids contains the barcode sequence based on the plurality of sequence similarities.

In some embodiments, the reference nucleic acid further comprises a second context sequence and the scoring region further comprises no more than a second threshold of nucleotides of the second context sequence. In some embodiments, prior to generating the plurality of alignments in step (i), generating a plurality of initial alignments between each of the plurality of target nucleic acids and an initial region of the reference nucleic acid that contains at least the barcode sequence and the first context sequence, wherein generating the plurality of alignments in step (i) is performed based on the plurality of initial alignments, and wherein the segment of the reference nucleic acid is the scoring region of the reference nucleic acid. In some embodiments, the plurality of target nucleic acids comprises at least 8, 16, 32, 64, 96, 192, 288, 384, or 480 target nucleic acids, and wherein each target nucleic acid comprises a discrete sequence or is from a discrete human patient.

In some aspects, the disclosure provides a method comprising using at least one computer hardware processor to perform:
(i) generating an alignment between at least a segment of a target nucleic acid and at least a segment of a reference nucleic acid, wherein the reference nucleic acid comprises a barcode sequence and a first context sequence,
(ii) determining a sequence similarity between a scoring region of the reference nucleic acid and a corresponding segment of the target nucleic acid, wherein the corresponding segment is identified based on the alignment, wherein the scoring region comprises at least a portion of the barcode sequence; and
(iii) determining whether the target nucleic acid comprises the barcode sequence based on the sequence similarity between the target nucleic acid and the scoring region of the reference nucleic acid.

In some embodiments, the reference nucleic acid further comprises a second context sequence.

In some embodiments, the method further comprises obtaining sequencing data from the plurality of target nucleic acids prior to step (i).

The segment of the reference nucleic acid or the segment of each of the plurality of reference nucleic acids may comprise the barcode sequence, at least a portion of the first context sequence, and/or at least a portion of the second context sequence. In some embodiments, the length of the segment of the reference nucleic acid or the segment of each of the plurality of reference nucleic acids is 25-50, 50-150, 100-200, 150-300, or 250-500 nucleotides. In some embodiments, the length of the barcode sequence is 4, 5, 6, 7, 8, 9, 10, 11, 12, 13, 14, 15, 15-20, or 20-25 nucleotides. In some embodiments, the length of the first context sequence is 5-10, 10-15, 15-20, 20-25, or 25-50 nucleotides. In some embodiments, the length of the second context sequence is 5-10, 10-15, 15-20, 20-25, or 25-50 nucleotides. In some embodiments, the first threshold number is 1, 2, 3, 4, 5, 6, 7, 8, 9, or 10. In some embodiments, the second threshold number is 0, 1, 2, 3, 4, 5, 6, 7, 8, 9, or 10. In some embodiments, the ratio of the first threshold number relative to the length of the barcode sequence is less than or equal to 1:1, 1:2, 1:3, 1:4, 1:5, 1:6, 1:7, 1:8, 1:9, or 1:10. In some embodiments, the ratio of the second threshold number relative to the length of the barcode sequence is less than or equal to 1:1, 1:2, 1:3, 1:4, 1:5, 1:6, 1:7, 1:8, 1:9, or 1:10.

In some embodiments, the at least one and no more than a first threshold number of nucleotides of the first context sequence in the scoring region are contiguous with the barcode sequence. In some embodiments, the no more than a second threshold number of nucleotides of the second context sequence in the scoring region are contiguous with the barcode sequence.

In some embodiments, the scoring region comprises 1-10 nucleotides of the first context sequence and 0-10 nucleotides of the second context sequence. In some embodiments, the scoring region comprises one nucleotide of the first context sequence and one nucleotide of the second context sequence.

In some embodiments, generating an alignment comprises generating data encoding an association between (a) the at least a segment of the target nucleic acid and the at least a segment of the reference nucleic acid; (b) the at least a segment of the target nucleic acids and the at least a segment of each of the plurality of reference nucleic acids; or (c) the at least a segment of each of the plurality of target nucleic acids and the at least a segment of the reference nucleic acid.

Determining the sequence similarity may comprise determining a score indicative of how many nucleotides of the target nucleic acid are aligned to similar nucleotides in the scoring region of the reference nucleic acid. In some embodiments, determining the sequence similarity comprises determining a percentage of nucleotides of the target nucleic acid that are aligned to similar nucleotides in the scoring region of the reference nucleic acid. In some embodiments, determining the sequence similarity comprises determining a score indicative of how many nucleotides of the target nucleic acid are aligned to identical nucleotides in the scoring region of the reference nucleic acid. In some embodiments, determining the sequence similarity comprises determining the percentage of nucleotides of the target nucleic acid that are aligned to identical nucleotides in the scoring region of the reference nucleic acid.

In some embodiments, barcodes are used in a combinatorial fashion, wherein more than one barcode is used to identify the origin. For example, the use of two instances of 96 barcodes in a combination provides 9216 identifiers, and the use of two instances of 384 barcodes provides 147456 identifiers.

The target nucleic acid or plurality of target nucleic acids may be amplified prior to step (i) of the method (e.g., using loop-mediated isothermal amplification (LAMP), polymerase chain reaction (PCR), multiple displacement amplification, rolling circle amplification (RCA), or ligase chain reaction). The amplification step may carried out to amplify RNA nucleic acid, such as RT-LAMP. LAMP and RT-LAMP methods of amplification are disclosed in WO01/77317, WO02/24902 and WO01/34790, hereby incorporated by reference in their entirety.

The target nucleic acid or at least one of the plurality of target nucleic acids may be from a human or veterinary patient. In some embodiments, the target nucleic acid or at least one of the plurality of target nucleic acids is indicative of disease or a genetic trait or marker. In some embodiments, identification of the barcode sequence in the target nucleic acid indicates that the patient associated with that barcode has or has had an infection (e.g., a viral or bacterial infection). In some embodiments, an infection is a SARS-CoV-2 infection. The target nucleic acid may comprise at least a segment of a gene associated with a SARS-CoV-2 infection (e.g., a SARS-CoV-2 ORF1a, SARS-CoV-2 envelope, or SARS-CoV-2 nucleocapsid gene). The origin nucleic acid may be derived from plants, animals, fungi, protists, archaea, or bacteria. The origin nucleic acid may be viral and comprise RNA.

In some embodiments, the method further comprises determining that the patient associated with the barcode sequence does not have an infection when a nucleic acid containing the barcode sequence is not detected.

The sequencing data for the target nucleic acid or plurality of nucleic acids may be obtained from measurement of a nucleic acid or plurality of nucleic acids using a variety of different sequencing methods, such as single molecule sequencing, sequencing by synthesis, or pyrosequencing. The detection means may be electrical or optical. Examples of single molecule sequencing include nanopore sequencing, and sequencing using a zero-mode waveguide such as SMRT sequencing using devices developed by Pacific Biosciences of California Inc., such as disclosed in WO2007/002893 and WO2009/120372. Examples of nanopore sequencing devices are disclosed in WO2015/055981, WO2014/064443, WO2017/149316, and, WO2019/002893, WO2015/110813 and WO2014/135838, hereby incorporated by reference in their entirety. Examples of sequencing by synthesis include ion semiconductor sequencing developed by Ion Torrent such as disclosed in WO2009/158006, sequencing based on fluorophore-labelled dNTPs with reversible terminator elements as developed by Illumina such as disclosed in WO00/18957, semiconductor chip-based single-molecule sequencing technology as developed by Roswell Technologies such as disclosed in WO16/210386 and sequencing by synthesis methods as developed by Genia Technologies, such as disclosed in WO2015/148402.

In some embodiments, the target nucleic acid and/or plurality of nucleic acids are 1 kilobase or longer.

Some aspects of the disclosure provide a kit comprising a plurality of nucleic acids, wherein each of the plurality comprises a respective barcode having fewer than ten nucleotides and at least one fixed context sequence. In some embodiments, each of the plurality comprises one fixed context sequence on each side of the barcode. In some embodiments, each of the plurality further comprises a primer sequence, and wherein the primer sequence is complementary to a segment of a target nucleic acid. In some embodiments, the at least one fixed context sequence comprises at least a part of the primer sequence. In some embodiments, the kit further comprises a polymerase.

### DESCRIPTION OF THE DRAWINGS

FIGS. 1A-1C provide schematics of exemplary methods of the disclosure.

FIG. 2 provides representative depictions of exemplary methods to identify whether a target nucleic acid (query) comprises a particular barcode sequence. Method A involves generating an alignment between the query and a context-barcode-context sequence and determining a sequence similarity between the query and a scoring region that includes the barcode and segments of the context sequences. Method B involves generating an initial alignment between the query and a context-barcode-context sequence, generating an alignment based on the initial alignment between the query and a scoring region that includes the barcode and segments of the context sequences, and then determining a sequence similarity between the query and the scoring region.

FIG. 3 provides a graph showing the number of correct and incorrect identifications of a barcoded target nucleic acid from 1000 simulated examples using reference nucleic acids comprising a fixed context sequence and a BC05 barcode. The simulated target nucleic acid was aligned and scored against a set of eight barcodes comprising either the full sequence or the barcode sequence with 0, 1, 2, or 3 flanking nucleotides.

FIG. 4 provides a graph showing that the total and relative counts of incorrect and correct determinations of whether a target nucleic acid comprises a SARS-CoV-2 sequence in a multiplexed experiment comprising positive and negative samples. The counts vary depending on the number of flanking nucleotides on either side of the barcode sequence used for scoring, and on the chose edit distance threshold. For example, the number of incorrect counts can be reduced by ˜20% by increasing the number of flanking nucleotides used for scoring from 0 to 1 (with an allowed edit-distance from 1), while only reducing the number of correct counts by ˜4%.

FIG. 5 provides a graph showing the number of correct and incorrect identifications of a barcoded target nucleic acid from 1000 simulated examples using reference nucleic acids comprising a fixed context sequence and a BC05 barcode. The simulated target nucleic acid was aligned against a segment of the reference nucleic acids and then scored against a set of eight barcodes comprising either the full sequence of the reference nucleic acid or the barcode sequence with 0, 1, 2, or 3 flanking nucleotides.

FIG. 6 provides an example of a preparation of a library of nucleic acids comprising barcodes for use in a multiplexed loop-mediated isothermal amplification-based experiment (LamPORE).

FIGS. 7A-7C provide schematics showing multimeric sequencing reads aligned to the SARS-CoV-2 genome. FIG. 7A shows sequencing reads that correspond to all three assayed loci of the genome. FIG. 7B shows a focused view on a single read aligned to the AS1 target in ORF1a, showing the alternating orientation of unequal consecutive repeating units. FIG. 7C shows position of 10-nucleotide barcodes positioned along the SARS-CoV-2 genome.

FIGS. 8A-8B provide graphs showing that valid reads and primer artifacts can be distinguished using an alignment. FIG. 8A shows valid reads consist of inverted repeats that align across the majority of the target region. FIG. 8B shows primer artifacts align as short segments interspersed with gaps.

FIG. 9 provides a graph displaying a selection of pairs of high-performing forward inner primer (FIP) barcodes and barcodes added during library preparation by the rapid barcoding kit (RB K). The displayed numbers indicate the quantity of template copies added to a reaction.

FIGS. 10A-10B provide measures of performance and threshold selection in a multiplexed LamPORE experiment. FIG. 10A shows receiver operating characteristic (ROC) curves demonstrating the true and false positive rates at varying SARS-CoV-2 target read count thresholds, for the sum of read counts from all three SARS-CoV-2 targets and each individual SARS-CoV-2 target. FIG. 10B shows a correlation between the F1 score and the read-count threshold that can be used to identify the optimal read count threshold for identifying a SARS-CoV-2 positive sample.

FIG. 11 provide schematics of an illustrative system that may be used in implementing some embodiments of the disclosure.

### DETAILED DESCRIPTION

Described herein are methods, kits, systems, and computer readable storage medium storing processor executable instructions for detection of nucleic acids (e.g., a target nucleic acid) having a particular barcode sequence. The inventors have discovered a novel method of determining whether a target nucleic acid comprises a particular barcode sequence that can be performed rapidly and with a high degree of accuracy (e.g., identification of true positives). This method, in some embodiments, involves the use of at least one sequence alignment between a target nucleic acid (e.g., at least a segment of a target nucleic acid) followed by a determination of sequence similarity between a scoring region of the reference nucleic acid and a corresponding segment of the target nucleic acid to enable determination of whether the target nucleic acid comprises a particular barcode, wherein the scoring region includes the particular barcode sequence and flanking nucleotides belonging to at least one context sequence (e.g., fixed context sequence). In some embodiments, the method is used to identify a target nucleic acid having a particular barcode in order to determine whether a subject (e.g., a human patient) with whom the particular barcode is associated has a disease or infection (e.g., a SARS-CoV-2 infection).

The methods of the disclosure involve complex computations, namely generating sequence alignments and determining sequence similarities between two nucleic acid segments, that necessitate the use of a system (e.g., a computer system as described by FIG. 11). The complex computations may be done sequentially or combined in a single act or algorithm. In some embodiments, the sequence alignments are performed between target nucleic acids that are hundreds or even thousands of nucleotides in length and a reference nucleic acid. In some further embodiments, the methods of the disclosure are multiplexed methods (e.g., comprising a plurality of target nucleic acids and/or reference nucleic acids, wherein the plurality may number hundreds or thousands).

Furthermore, the methods described herein reduce incorrect assignment of barcodes, particularly relative to methods of assigning barcodes that were known in the art. Incorrect assignment may be caused by sequencing errors, spurious alignments, alignment artifacts, or other issues either individually or in combination. The methods described address spurious alignments and alignment artifacts around edges of barcodes in the presence of sequencing errors.

Employing barcode identification techniques described in this application also provides an improvement to sequencing technology and computer technology. Sequencing data that is correctly assigned to an origin-specific sample (e.g., a particular patient sample) by correctly identifying a barcode sequence reduces or eliminates errors in downstream applications (e.g., identifying the presence of one or more indicia of a infection, identifying one or more biomarkers indicative of a disease or condition, recommending and/or administering an appropriate therapy to a patient, etc.). Also, correctly identifying barcode sequences can prevent computationally expensive processes from being executed by avoiding unnecessary interpretation and analysis of complex sequencing data that is associated with an incorrect sample source. This can reduce or eliminate wasteful use of computing resources, saving processing power, memory, and networking resources (which is an improvement to computing technology in addition to being an improvement to sequencing technology). Reducing errors in barcode identification will also reduce waste of resources at a laboratory that processes multiple samples, by freeing up equipment for processing biological samples that are correctly associated with the sample sources and avoiding duplicating and/or repeating experimental analysis for samples that produced incorrect or inconclusive results due to errors in barcode identification. In addition, sequence data for which the source is correctly identified can be useful to select more effective therapies for a patient, improve ability to determine whether one or more cancer therapies will be effective if administered to the patient, improve the ability to identify clinical trials in which the subject may participate, and/or improvements to numerous other prognostic, diagnostic, and clinical applications.

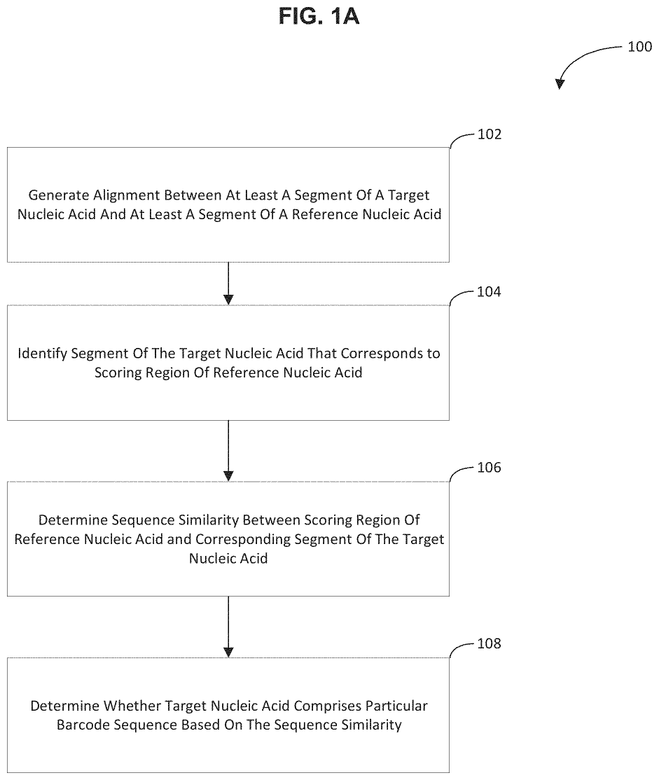

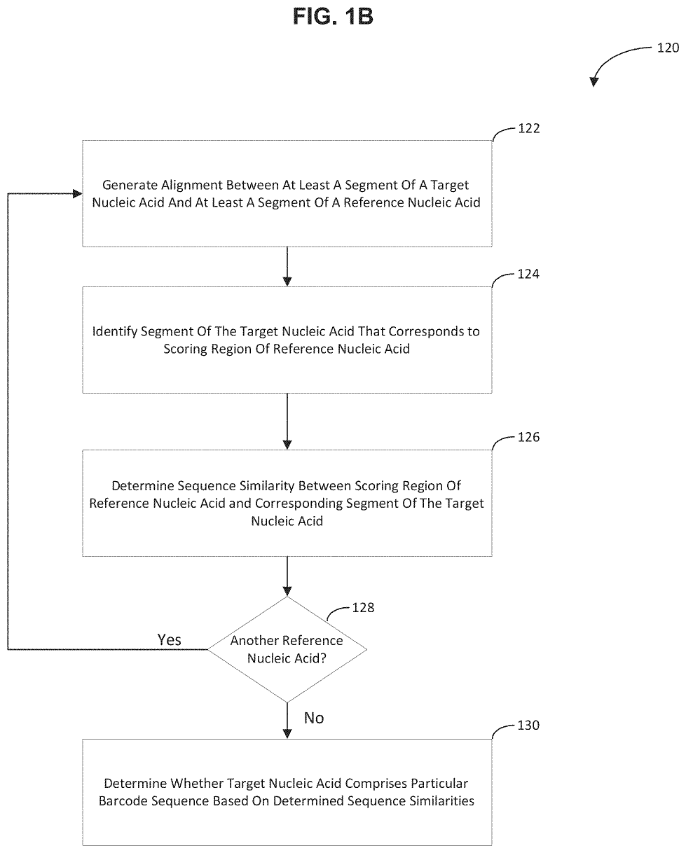

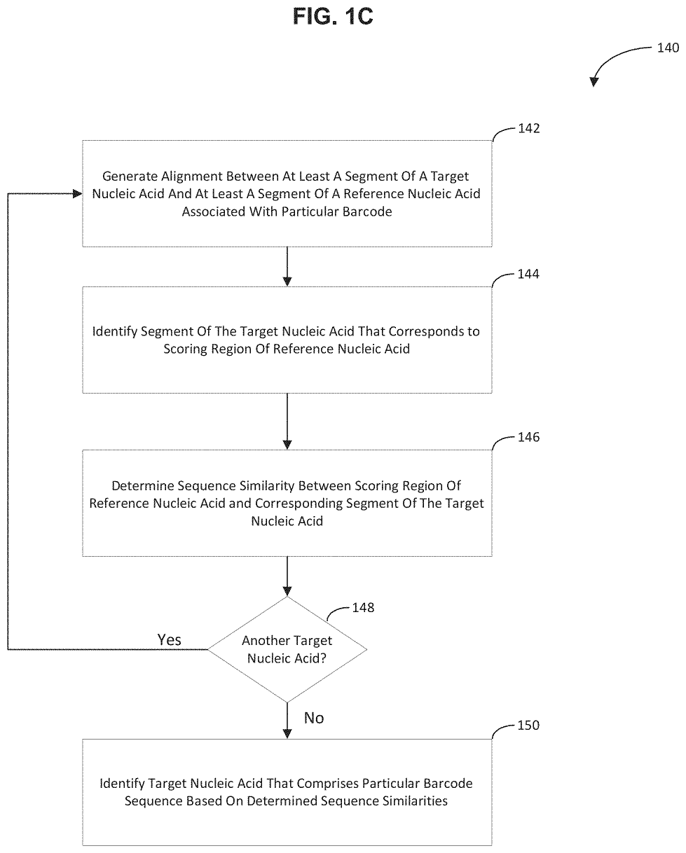

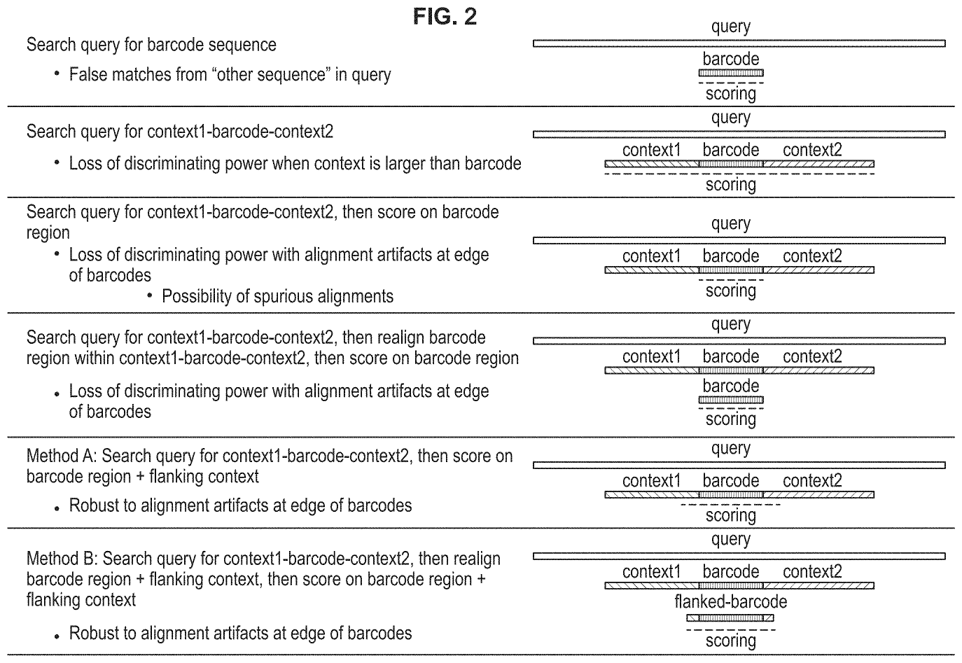

FIG. 1A is a flowchart of an illustrative process 100 for determining whether a target nucleic acid comprises a particular barcode sequence. Process 100 may be performed by any suitable computing device(s) including any of the device(s) described herein including with reference to FIG. 11.

As shown in FIG. 1A, process 100 begins at act 102, where an alignment between at least a segment of a target nucleic acid and at least a segment of a reference nucleic acid comprising a particular barcode sequence is generated. Next, process 100 proceeds to act 104, where, based on the alignment generated at act 102, a segment of the target nucleic acid that corresponds to a scoring region of the reference nucleic acid (e.g., a scoring region comprising the particular barcode sequence and at least one and no more than a first threshold number of nucleotides of a context sequence) is identified. Next, at act 106, a sequence similarity is determined between the scoring region of the reference nucleic acid and the corresponding segment of the target nucleic acid. Finally, at act 108, the sequence similarity is used to determine whether the target nucleic acid comprises a particular barcode sequence.

FIG. 1B is a flowchart of an illustrative process 120 for determining whether a target nucleic acid comprises a particular barcode sequence. Process 120 may be performed by any suitable computing device(s) including any of the device(s) described herein including with reference to FIG. 11.

As shown in FIG. 1B, process 120 begins at act 122, where an alignment between at least a segment of a target nucleic acid and at least a segment of a reference nucleic acid comprising a barcode sequence is generated. Next, process 120 proceeds to act 124, where, based on the alignment generated at act 122, a segment of the target nucleic acid that corresponds to a scoring region of the reference nucleic acid (e.g., a scoring region comprising the barcode sequence and at least one and no more than a first threshold number of nucleotides of a context sequence) is identified. Next, at act 126, a sequence similarity is determined between the scoring region of the reference nucleic acid and the corresponding segment of the target nucleic acid. Next, at act 128, the operator (e.g., a suitable computing device) of the process determines whether to replicate acts 122-126 using the same target nucleic acid and another reference nucleic acid comprising a different barcode sequence. Acts 122-128 are iterated at least once and as many times as needed or desired by the operator. Following act 128, if there are no additional reference nucleic acids, then finally, at act 130, the sequence similarities are used to determine whether the target nucleic acid comprises a particular barcode sequence.

FIG. 1C is a flowchart of an illustrative process 140 for identifying a target nucleic acid comprising a particular barcode sequence. Process 140 may be performed by any suitable computing device(s) including any of the device(s) described herein including with reference to FIG. 11.

As shown in FIG. 1C, process 140 begins at act 142, where an alignment between at least a segment of a target nucleic acid and at least a segment of a reference nucleic acid comprising a particular barcode sequence is generated. Next, process 140 proceeds to act 144, where, based on the alignment generated at act 142, a segment of the target nucleic acid that corresponds to a scoring region of the reference nucleic acid (e.g., a scoring region comprising the particular barcode sequence and at least one and no more than a first threshold number of nucleotides of a context sequence) is identified. Next, at act 146, a sequence similarity is determined between the scoring region of the reference nucleic acid and the corresponding segment of the target nucleic acid. Next, at act 148, the operator (e.g., a suitable computing device) of the process determines whether to replicate acts 142-146 using another target nucleic acid (e.g., from a different subject, e.g., human patient) and the same reference nucleic acid. Acts 142-148 are iterated at least once and as many times as needed or desired by the operator. Following act 148, if there are no additional target nucleic acids, then finally, at act 150, the to identify which target nucleic acid from the plurality of target nucleic acids comprises the particular barcode sequence.

### Generating an Alignment

In some embodiments, generating an alignment comprises generating data encoding an association between segment of two nucleic acids (e.g., a target nucleic acid and a reference nucleic acid). In some embodiments, an alignment between two nucleic acid sequences may include any information indicative of an association between the two nucleic acid sequences. In some embodiments, the information indicative of the association between two sequences may indicate corresponding segments of the two sequences (e.g., by indicating, for a first segment of a first sequence, a second segment of the second sequence to which the first segment corresponds). This may be done in any suitable way. For example, an alignment may comprise information indicating, for a first segment of the first sequence, the position(s) in the second sequence of at least some nucleotides of a second segment that corresponds to the first segment. The positions may be specified in any suitable way (e.g., first and last positions, first position and an offset, all positions, etc.), as aspects of the disclosure described herein are not limited in this respect.

In some embodiments, corresponding segments of two nucleic acid sequences may be identical or, if not identical, may have some similarity. For example, corresponding sequence segments may have the same nucleotides at some (e.g., at least a threshold percentage) or all of the corresponding positions. As another example, associated segments may have complementary nucleotides at some (e.g., at least a threshold percentage) or all of the corresponding positions (e.g., in this context, “G” is a complementary nucleotide to a “C” and an “A” is a complementary nucleotide to a “T”).

In some embodiments, generating the alignment comprises using a scoring function based on expected properties of the sequence data. In some embodiments, the expected properties of the sequence data comprise features corresponding to platform specific error modalities. In some embodiments, the expected properties of the sequence data comprise features corresponding to the variations and/or distribution and/or positions of bases within the expected barcode sequences.

In some embodiments, an alignment between two nucleotide sequences may be stored in any suitable non-transitory computer-readable storage medium (e.g., a volatile memory or a non-volatile memory), using any suitable data structure(s), and in any suitable format, as aspects of the disclosure described herein are not limited in this respect.

In some embodiments, generating an alignment between two nucleotide sequences may be performed one or more sequence alignment algorithms. In some embodiments, a dynamic programming-based sequence alignment algorithm may be used. Non-limiting examples of dynamic programming-based sequence alignment algorithms include the Needleman-Wunsch algorithm (e.g., as described in Needleman, Saul B. & Wunsch, Christian D. (1970). “A general method applicable to the search for similarities in the amino acid sequence of two proteins”. Journal of Molecular Biology. 48 (3): 443-53. doi:10.1016/0022-2836(70)90057-4. PMID 5420325, which is incorporated in its entirety herein) and the Smith-Waterman algorithm (e.g., as described in Smith, Temple F. & Waterman, Michael S. (1981). “Identification of Common Molecular Subsequences” (PDF). Journal of Molecular Biology. 147 (1): 195-197. CiteSeerX 10.1.1.63.2897. doi:10.1016/0022-2836(81)90087-5. PMID 7265238, which is incorporated by reference in its entirety herein). However, any other suitable sequence alignment algorithm(s) may be used (e.g., FASTA, BLAST, brute force, dot-matrix alignments, etc.), as aspects of the technology described herein are not limited in this respect.

### Determining a Sequence Similarity

In some embodiments, determining a sequence similarity comprises generating data encoding the sequence similarity (e.g., sequence identity) between corresponding segments of two nucleic acids (e.g., a scoring region of a reference nucleic acid and a corresponding segment of a target nucleic acid). In some embodiments, sequence similarity between corresponding segments of two nucleic acid sequences is determined based on the presence of identical nucleotides at some (e.g., at least a threshold percentage) or all of the corresponding positions of the two nucleic acid sequences. In some embodiments, sequence similarity between corresponding segments of two nucleic acid sequences is determined based on the presence of purines (e.g., adenine or guanine) at some (e.g., at least a threshold percentage) or all of the corresponding positions. In some embodiments, sequence similarity between corresponding segments of two nucleic acid sequences is determined based on the presence of pyrimidines (e.g., thymine or cytosine) at some (e.g., at least a threshold percentage) or all of the corresponding positions.

In some embodiments, determining a sequence similarity involves determining a percentage of nucleotides of a segment of first nucleic acid (e.g., target nucleic acid) that are aligned to similar nucleotides (e.g., identical nucleotides) in a segment of a second nucleic acid (e.g., the scoring region of the reference nucleic acid). In some embodiments, the percentage of nucleotides in the segment of the first nucleic acid that are aligned to similar nucleotides in the segment of the second nucleic acid is at least 50%, 60%, 70%, 80%, 90%, 95%, or 99%.

In some embodiments, determining a sequence similarity comprises determining a score indicative of how many nucleotides of a segment of first nucleic acid (e.g., target nucleic acid) are aligned to similar nucleotides (e.g., identical nucleotides) in a segment of a second nucleic acid (e.g., the scoring region of the reference nucleic acid). In some embodiments, the number of nucleotides in the segment of the first nucleic acid that are aligned to similar nucleotides in the segment of the second nucleic acid is at least 2, 3, 4, 5, 6, 7, 8, 9, 10, 11, 12, 13, 14, 15, 16, 17, 18, 19, or 20. In some embodiments, the number of nucleotides in the segment of the first nucleic acid that are aligned to similar nucleotides in the segment of the second nucleic acid is 2, 3, 4, 5, 6, 7, 8, 9, 10, 11, 12, 13, 14, 15, 16, 17, 18, 19, or 20.

In some embodiments, determining the sequence similarity comprises determining a score indicative of how many nucleotides of a segment of first nucleic acid (e.g., target nucleic acid) are not aligned to a segment of a second nucleic acid (e.g., the scoring region of the reference nucleic acid). In some embodiments, determining the sequence similarity comprises determining a score indicative of the number of insertions and deletions in the alignment between the at least a segment of the segment of first nucleic acid (e.g., target nucleic acid) and the segment of a second nucleic acid (e.g., the scoring region of the reference nucleic acid). In some embodiments, determining the sequence similarity comprises determining the edit distance between the segment of first nucleic acid (e.g., target nucleic acid) and the segment of a second nucleic acid (e.g., the scoring region of the reference nucleic acid). In some embodiments, determining the sequence similarity comprises determining an alignment score between the segment of first nucleic acid (e.g., target nucleic acid) and the segment of a second nucleic acid (e.g., the scoring region of the reference nucleic acid) using a scoring function based on expected properties of the sequence data. In some embodiments, the expected properties of the sequence data comprise features corresponding to platform specific error modalities. In some embodiments, the expected properties of the sequence data comprise features corresponding to the variations and/or distribution and/or positions of bases within the expected barcode sequences.

In some embodiments, determining a sequence similarity in the context of the methods described herein involves determining a sequence similarity between a scoring region of a reference nucleic acid and a corresponding segment of a target nucleic acid. A scoring region of a reference nucleic acid may comprise at least a portion of barcode sequence and at least one and no more than a first threshold number of nucleotides of a first context sequence. In some embodiments, the scoring region further comprises no more than a second threshold of nucleotides of a second context sequence.

In some embodiments, determining a sequence similarity comprises using a scoring function based on expected properties of the sequence data. In some embodiments, the expected properties of the sequence data comprise features corresponding to platform specific error modalities. In some embodiments, the expected properties of the sequence data comprise features corresponding to the variations and/or distribution and/or positions of bases within the expected barcode sequences.

A barcode sequence may be 2, 3, 4, 5, 6, 7, 8, 9, 10, 11, 12, 13, 14, 15, 16, 17, 18, 19, or 20 nucleotides in length. In some embodiments, a barcode sequence is 4-25, 4-20, 4-15, 4-10, 5-25, 5-20, 5-25, 5-10, 10-25, 10-20, 10-15, 15-25, or 20-25 nucleotides in length. In some embodiments, a barcode sequence is 2, 3, 4, 5, 6, 7, 8, 9, 10, 11, 12, 13, 14, 15, 16, 17, 18, 19, or 20 nucleotides in length. In some embodiments, a barcode sequence is 4-25, 4-20, 4-15, 4-10, 5-25, 5-20, 5-25, 5-10, 10-25, 10-20, 10-15, 15-25, or 20-25 nucleotides in length. In some embodiments, a scoring region comprises the entire length of the barcode sequence. In some embodiments, a scoring region comprises 50-75%, 50-100%, 60-80%, 70-100%, or 80-95% of the barcode sequence. In some embodiments, a scoring region comprises a contiguous portion of a barcode sequence. In some embodiments, a scoring region comprises a non-contiguous portion of a barcode sequence.

A context sequence (the first and/or second context sequences) may be 5-10, 10-15, 15-20, 20-25, 20-50, 30-60, 25-50, 30-75, 50-75, 50-100, or 75-150 nucleotides in length. The first threshold number may be 1, 2, 3, 4, 5, 6, 7, 8, 9, 10, 11, 12, 13, 14, 15, 16, 17, 18, 19, 20, 21, 22, 23, 24, or 25. The second threshold number is may be 0, 1, 2, 3, 4, 5, 6, 7, 8, 9, 10, 11, 12, 13, 14, 15, 16, 17, 18, 19, 20, 21, 22, 23, 24, or 25.

In some embodiments, the ratio of the first threshold number relative to the length of the barcode sequence is less than 1:1, 1:2, 1:3, 1:4, 1:5, 1:6, 1:7, 1:8, 1:9, 1:10, 1:12, 1:15, or 1:20. In some embodiments, the ratio of the first threshold number relative to the length of the barcode sequence is equal to about 1:1, 1:2, 2:3, 1:3, 1:4, 1:5, 1:6, 1:7, 1:8 1:9, 1:10, 1:12, 1:15, or 1:20. In some embodiments, the ratio of the second threshold number relative to the length of the barcode sequence is less than 1:1, 1:2, 1:3, 1:4, 1:5, 1:6, 1:7, 1:8, 1:9, 1:10, 1:12, 1:15, or 1:20. In some embodiments, the ratio of the second threshold number relative to the length of the barcode sequence is equal to about 1:1, 1:2, 2:3, 1:3, 1:4, 1:5, 1:6, 1:7, 1:8 1:9, 1:10, 1:12, 1:15, or 1:20. In some embodiments, the ratio of the first threshold number relative to the length of the barcode sequence is 1:10; and the ratio of the second threshold number relative to the length of the barcode sequence is 1:10. For example, in some embodiments, if the length of the barcode is 10 nucleotides, then the first threshold number may be 1 (i.e., ratio of the first threshold number relative to the length of the barcode sequence is 1:10).

In some embodiments, the at least one and no more than a first threshold number of nucleotides of the first context sequence in the scoring region are contiguous with the barcode sequence. In some embodiments, the no more than a second threshold number of nucleotides of the second context sequence in the scoring region are contiguous with the barcode sequence. In some embodiments, the at least one and no more than a first threshold number of nucleotides of the first context sequence in the scoring region are not contiguous with the barcode sequence. In some embodiments, the no more than a second threshold number of nucleotides of the second context sequence in the scoring region are not contiguous with the barcode sequence.

In some embodiments, when the first threshold number is two or more, the two or more nucleotides are contiguous with one another. In some embodiments, when the second threshold number is two or more, the two or more nucleotides are contiguous with one another. In some embodiments, when the first threshold number is two or more, the two or more nucleotides are not contiguous with one another. In some embodiments, when the second threshold number is two or more, the two or more nucleotides are not contiguous with one another.

In some embodiments, a scoring region comprises 1-10, 1-5, 5-10, 5-15, or 5-20 nucleotides of a first context sequence. In some embodiments, a scoring region comprises 0-10, 0-5, 5-10, 5-15, or 5-20 nucleotides of the second context sequence. In some embodiments, a scoring region comprises one, two, three, or four nucleotide(s) of a first context sequence and one, two, three, or four nucleotide(s) of a second context sequence.

### Barcode Sequences

A barcode sequence is a variable nucleic acid sequence that is origin- and/or sample-specific. A barcode sequence may be used to uniquely tag or link a target nucleic acid to a specific subject (e.g., a human or veterinary patient).

In some embodiments, a barcode sequence is short (e.g., for chemistry-driven reasons, e.g., ease of synthesis and purification). In some embodiments, the methods described herein utilize a large number of barcode sequences (e.g., more than 2, 5, 10, 15, etc.) in a multiplexed assay to tag or identify a large number of samples. In some embodiments, a barcode sequences are utilized within different contexts. In some embodiments, barcode sequences are utilized within the same context. In some embodiments, a barcode sequence can share nucleotides with surrounding (e.g., contiguous) context sequence.

In some embodiments, a barcode sequence is 4, 5, 6, 7, 8, 9, 10, 11, 12, 13, 14, 15, 15-20, or 20-25 nucleotides in length. In some embodiments, a barcode sequence comprises less than 10%, 15%, 20%, 25%, 30%, or 50% of the entire number of nucleotides in a target sequence.

In some embodiments, a multiplexed sample comprising more than one nucleic acid comprising a barcode sequence includes at least 2, 4, 8, 16, 32, 64, 96, 192, 288, 384, 480 or 9216 nucleic acids, each of which comprises a respective and unique barcode sequence (e.g., reference nucleic acids comprising respective and unique barcodes). In some embodiments, there could be single (one barcode per read), or combinatorial (e.g., dual barcoding or two barcodes per read).

### Context Sequence

A context sequence is generally a fixed (e.g., constant) nucleic acid sequence that is present on a target nucleic acid comprising a barcode sequence. In some embodiments, a fixed context sequence consists of a single nucleic acid sequence that is identical across multiple target nucleic acids, wherein each of the multiple target nucleic acids comprises its own respective barcode sequence. Context sequences are typically larger than the barcode and can be on one or both sides of barcode. In some embodiments, a context sequence is contiguous with the barcode. In some embodiments, a context sequence is not contiguous with the barcode. A context sequence (e.g., a first and/or second context sequences) may be 5-10, 10-15, 15-20, 20-25, 20-50, 30-60, 25-50, 30-75, 50-75, 50-100, or 75-150 nucleotides in length.

In some embodiments, a context sequence comprises at least a part of a primer sequence. In some embodiments, a context sequence comprises at least a part of an amplification primer. In some embodiments, a context sequence comprises at least a part of a sequencing primer. In some embodiments, a context sequence comprises at least a part of a universal primer.

In some embodiments, a context sequence comprises a consistent and identical sequence (e.g., multiple or all nucleic acids in a multiplexed sample comprise identical context sequences). In some embodiments, a context sequence comprises sequence that is consistent in content but variable in length (e.g. a polyA of variable length, e.g., a polyA tail on a transcript). In some embodiments, a context sequence is consistent in length but has a pattern of variation. For example, in some embodiments, a context sequence may always start and/or end with the same nucleotides and/or have consistent nucleotides at specific positions (e.g., the third base is always A).

In some embodiments, the context sequence comprises a technology specific sequence. By way of non-limitative examples, a technology specific sequence may be part of a sequencing adapter that is use to hybridise to a substrate or otherwise immobilise DNA, a leader sequence, an enzyme binding sequence, an enzyme stalling sequence, a registration sequence, a calibration sequence, a ligatable sticky-end, or a transposable element.

### Target Nucleic Acids

A target nucleic acid, in some embodiments, is a nucleic acid that comprises a particular barcode. Accordingly, a target nucleic comprising a particular barcode is a target nucleic acid that is an origin-specific nucleic acid. An origin-specific nucleic acid may be associated with a particular subject (e.g., a human or veterinary patient). By way of example, an origin-specific nucleic acid may be associated with a particular subject, for example, a human or animal subject (for example a human or veterinary patient), or a plant subject. An origin-specific nucleic acid may be associated with an environmental sample. An origin-specific nucleic acid may be derived from a synthetic nucleic acid sequence, for example a synthetic nucleic acid sequence produced as part of an experimental or industrial process or assay, for example a synthetic nucleic acid sequence produced using a DNA data-storage system. An origin-specific nucleic acid may be DNA or RNA.

In some embodiments, a target nucleic acid (e.g., a target nucleic acid comprising a particular barcode) comprises a nucleotide sequence corresponding to a gene, a segment of a gene, and/or a regulatory element of a gene (e.g., a promoter region). The gene may be associated with a disease, genetic trait, or marker. In some embodiments, the gene sequence is associated with a bacterial or viral infection. In some embodiments, the gene sequence is associated with a SARS-CoV-2 infection. In some embodiments, the gene sequence is a SARS-CoV-2 ORF1a, SARS-CoV-2 envelope, or SARS-CoV-2 nucleocapsid gene.

Accordingly, in some embodiments, detection of a target nucleic acid comprising a barcode and a gene, a segment of a gene, and/or a regulatory element of a gene (e.g., a promoter region) associated with a disease, genetic trait, or marker indicates that the subject associated with that particular barcode has or has had an infection (e.g., a viral infection, e.g., a SARS-CoV-2 infection).

In some embodiments, a multiplexed sample comprising a plurality of target nucleic acids includes at least 2, 4, 8, 16, 32, 64, 96, 192, 288, 384, 480 or 9216 target nucleic acids. In some embodiments, each target nucleic acid comprises a respective and unique barcode sequence. In some embodiment, each target nucleic acid comprises a discrete sequence or is from a discrete human patient.

In some embodiments, a target nucleic acid comprises a nucleotide sequence corresponding to a region of variant sequence. In some embodiments, this variant may be a single nucleotide polymorphism, a small insertion or deletion, or a larger structural variant. In some embodiments, identification of target nucleic acids may be used to estimate the proportion of a variant present in a particular sample.

In some embodiments, multiple copies of the target nucleic acid are present in the sequencing read. To increase specificity, where conflicting targets are detected, these reads may be discarded. Alternatively, multiple copies may be used to form a consensus sequence. In some embodiments, the consensus sequence may be used to call one or more sequence variants. In some embodiments sequence variants may be called from multiple sequence alignments to the target region, or from a multiple sequence alignment. In some embodiments, the consensus sequence may be used to further refine target classification, for example by discriminating between similar targets that differ by one or more regions of variant sequence.

### Barcode Configurations

In some embodiments, barcodes are used in a combinatorial fashion, wherein more than one barcode is used to identify the origin. For example, the use of two instances of 96 barcodes in a combination provides 9216 identifiers, and the use of two instances of 384 barcodes provides 147456 identifiers.

In some embodiments, barcodes are present at the start of a sequencing read. In some embodiments these barcodes are added prior to, or as part of addition of a sequencing adapter. Where barcodes are expected at the start of a read, to improve specificity, when those barcodes or their contexts are detected within the read, those reads can be discarded.

In some embodiments, barcodes are present at the end of a sequencing read. In some embodiments, barcodes are present within a sequencing read. In some embodiments the same barcode may appear multiple times within a sequencing read. In some embodiments a barcode at the start of a sequencing read and a barcode within the sequencing read are used in a combinatorial fashion.

In embodiments where multiple barcodes are present, the multiple barcodes can be used to further refine barcode calling specificity. By way of non-limitative example, in combinatorial barcoding, where not all combinations are expected, reads where an unexpected combination is detected can be discarded. Where multiple identical barcodes are expected within a read, or at the start and end of reads, reads where conflicting barcodes are detected may be discarded. Alternatively, multiple barcodes, and their flanking sequence, may be used to form a barcode consensus before classification. Alternatively, majority voting could be used to identify the barcode.

### Multiplexed Assay

In some embodiments, target sequences corresponding to more than one target are present. For example, multiple barcoded primers may be mixed to target multiple sequences or genes or regions of genes. In some embodiments multiple barcoded primers may share the same barcode for a given origin, with that barcode appearing in a different primer-dependent context.

In some embodiments multiple barcoded primers may have different barcodes for a given origin. In some embodiments one of the multiple targets may be interpreted as an in-assay control to indicate that amplification has occurred correctly and/or that the sample was collected correctly and/or for other control purposes.

### Amplification and Sequencing Methods

Nucleic acid sequencing data of a target nucleic acid comprising a barcode or a plurality of target nucleic acids comprising respective barcodes are often obtained prior to performance of the methods described herein. Sequencing data may be obtained using any method known to a skilled person in the art. In some embodiments, sequencing data are obtained from measurement of a nucleic acid or plurality of nucleic acids using a single molecule sequencing device, a nanopore sequencing device, a zero-mode waveguide, or sequencing by synthesis. In some embodiments, the sequencing data produces nucleic acid reads that are at least 0.5, 0.75, 1, 1.5, 2, 2.5, 3, 4, or 5 kilobases in length. In some embodiments, the sequencing data produces nucleic acid reads that are 0.5-1, 0.75-1.25, 1-1.5, 1.5-2.5, 2-4, 2.5-5, 3-5, 4-6, or 5-7 kilobases in length.

In some embodiments, nanopore sequencing involves the measurement of an electrical current as template nucleic acids pass through each pore on a flow-cell array. This measurement of electrical current can be used to determine the sequence identity of an unknown nucleic acid. Nanopore sequencing, which does not have a fixed run time, can be matched to the data requirements. As a consequence, data analysis can be performed in real time, and results can be returned very rapidly.

In some embodiments, to maintain the speed/rapidity advantages offered by nanopore sequencing, it is beneficial to use a correspondingly rapid method of library preparation to convert amplified nucleic acids into a form that is compatible with sequencing. In some embodiments, a rapid method of library preparation involves the use of a barcoded rapid library prep kit which uses a transposase to convert DNA to a barcoded library that is ready to sequence in approximately 10 minutes. In some embodiments, 96 barcodes are available, allowing prepared samples to be pooled for multiplexed sequencing.

In some embodiment, sequencing data may be obtained from measurement of a nucleic acid or plurality of nucleic acids using a variety of different sequencing methods, such as single molecule sequencing, sequencing by synthesis, or pyrosequencing. The detection means may be electrical or optical. Examples of single molecule sequencing include nanopore sequencing, and sequencing using a zero-mode waveguide such as SMRT sequencing using devices developed by Pacific Biosciences of California Inc., such as disclosed in WO2007/002893 and WO2009/120372. Examples of nanopore sequencing devices are disclosed in WO2015/055981, WO2014/064443, WO2017/149316, and, WO2019/002893, WO2015/110813 and WO2014/135838, hereby incorporated by reference in their entirety. Examples of sequencing by synthesis include ion semiconductor sequencing developed by Ion Torrent such as disclosed in WO2009/158006, sequencing based on fluorophore-labelled dNTPs with reversible terminator elements as developed by Illumina such as disclosed in WO00/18957, semiconductor chip-based single-molecule sequencing technology as developed by Roswell Technologies such as disclosed in WO16/210386 and sequencing by synthesis methods as developed by Genia Technologies, such as disclosed in WO2015/148402.

In some embodiments, a target nucleic acid comprising a barcode or a plurality of target nucleic acids comprising respective barcodes are amplified prior to performance of the methods described herein. Nucleic acids may be amplified using any method known to a skilled person in the art. In some embodiments, nucleic acids are amplified using loop-mediated isothermal amplification (LAMP), polymerase chain reaction, multiple displacement amplification, rolling circle amplification, or ligase chain reaction. In some embodiments, any technique known to a skilled person for adding a barcode to a target nucleic acid may be used. In some embodiments, a barcode is added to a target nucleic acid using chemical ligation or amplification techniques.

LAMP is a method of targeted isothermal amplification which can generate micrograms of product from tens of copies of a segment of a target nucleic acid, within 30 minutes at 65° C. Successful amplification is often inferred from a proxy measurement, such as increased turbidity, a color change or changes in fluorescence. However, although the LAMP reaction itself is very robust, these proxy measurements are less robust and can be affected by substances present in biological samples. It is also not uncommon to see a color change or increase in turbidity in no-template controls, arising from amplification of primer artefacts, which would lead to a false positive call. In some embodiments, a target nucleic acid is amplified using LAMP and then subsequently sequenced (e.g., using nanopore sequencing). On-target amplification events contain sequences that are not present in the primers and can be identified without ambiguity by alignment and subsequent scoring as described herein.

### Kits

The disclosure further provides a kit for use in a method of the disclosure. In some embodiments, a kit comprises a plurality of nucleic acids, wherein each of the plurality comprises a respective barcode having fewer than ten nucleotides and at least one fixed context sequence. In some embodiments, each of the plurality comprises one fixed context sequence on each side of the barcode. In some embodiments, each of the plurality comprises a primer sequence, wherein the primer sequence is complementary to a segment of a target nucleic acid. The primer sequence may overlap with one of the context sequences in part or in full. In some embodiments, the kit further comprises one or more other reagents or instruments which enable any of the embodiments of the method. Such reagents or instruments include one or more of the following: suitable buffer(s) (aqueous solutions), means to obtain a sample from a subject (such as a vessel or an instrument comprising a needle), means to amplify and/or express polynucleotides, a membrane or voltage or patch clamp apparatus. Reagents may be present in the kit in a dry state such that a fluid sample is used to resuspend the reagents. The kit may also, optionally, comprise instructions to enable the kit to be used in the method of the disclosure. The kit may comprise a magnet or an electromagnet. The kit may, optionally, comprise nucleotides and/or a polymerase. Example polymerases suitable for use in RT-LAMP amplification and PCR include Bst DNA Polymerases and Taq DNA Polymerases, examples of which are available from New England BioLabs Inc.

### Computational System

An illustrative implementation of a computer system 1100 that may be used in connection with any of the embodiments of the technology described herein is shown in FIG. 11. The computer system 1100 includes one or more processors 1110 and one or more articles of manufacture that comprise non-transitory computer-readable storage media (e.g., memory 1120 and one or more non-volatile storage media 1130). The processor 1110 may control writing data to and reading data from the memory 1120 and the non-volatile storage device 1130 in any suitable manner, as the aspects of the technology described herein are not limited in this respect. To perform any of the functionality described herein, the processor 1110 may execute one or more processor-executable instructions stored in one or more non-transitory computer-readable storage media (e.g., the memory 1120), which may serve as non-transitory computer-readable storage media storing processor-executable instructions for execution by the processor 1110.

Computing device 1100 may also include a network input/output (I/O) interface 1140 via which the computing device may communicate with other computing devices (e.g., over a network), and may also include one or more user I/O interfaces 1150, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

### EXAMPLES

### Example 1—Barcode-Only Alignments can be Spurious

A series of simulations were performed using a query nucleic acid (e.g., target nucleic acid) comprising a particular barcode, BC06 (TCTGCATCGT (SEQ ID NO: 3)). The data were analysed against a set of 8 barcodes (BC01-BC08). As demonstrated below, the query nucleic acid was aligned against (1) the full length of a reference nucleic acid comprising BC06 and then scored using the barcode of the reference and the corresponding segment of the query to provide a score of 99; (2) the correct BC06 barcode alone and then scored using the barcode to provide a score of 12; and (3) the incorrect BC04 barcode alone and then scored using the barcode to provide a score of 14. Notably, the incorrect barcode BC04 provided a better score than the correct barcode BC06 because of a spurious alignment.

However, when the scoring region was increased to include the barcode sequences and three contiguous nucleotides from context sequences on either side of the barcode, the correct barcode BC06 provided a score of 20 while the incorrect barcode BC04 provided a lower score of 18. This example demonstrates that the use of flanking nucleotides from the context sequence when scoring after an alignment provides discrimination of a correct barcode relative to an incorrect barcode.

```
*****Alignments to correct barcode:*****
Full sequence alignment to BC06: SEQ ID NO: 1
G-TTGTCG-CGAGACAGAAGGGTACA-C-GC-TCGTGTAGTTTTAACTAGGGCCGTATGAGAT
| |||||| ||||| ||||||||||. | || ||||||| ||||||||| |||||||||||||
GATTGTCGACGAGA-AGAAGGGTACTTCTGCATCGTGTA-TTTTAACTA-GGCCGTATGAGAT
Score = 99 SEQ ID NO: 2
Barcode alignment to BC06: SEQ ID NO: 1
GTTGTCGCGAGACAGAAGGGTACAC-GC-TCGTGTAGTTTTAACTAGGGCCGTATGAGAT
                                                | || ||||
-----------------------TCTGCATCGT---------------------------
Score = 12 SEQ ID NO: 3
Flanked barcode alignment to BC06 +/− 3 bases: SEQ ID NO: 1
GTTGTCGCGAGACAGAAGGGTACA-C-GC-TCGTGTAGTTTTAACTAGGGCCGTATGAGAT
                                          ||. | || |||||||
---------------------ACTTCTGCATCGTGTA------------------------
Score = 20 SEQ ID NO: 4
*****Alignments to miscalled barcode (barcode sequence):*****
Barcode alignment to BC04: SEQ ID NO: 1
GTTGTCGCGAGACAGAAGGGTACACGCTCGTGTAGTTTTAACTAGGGCCGTATGAGAT
        |||| |.|||
---CTCGC-ACACA--------------------------------------------
Score = 14 SEQ ID NO: 5
Flanked barcode alignment to BC04 +/− 3 bases: SEQ ID NO: 1
GTTGTCGCGAGACAGAAGGGTACACGCTCGTGTAGTTTTAACTAGGGCCGTATGAGAT
    |.|||| |.||||.|
ACTCTCGC-ACACAGTA-----------------------------------------
Score = 18 SEQ ID NO: 6
```

### Example 2—Barcode-Only Alignments can Steal Bases from Surrounding Context

A series of simulations were performed using a query nucleic acid (e.g., target nucleic acid) comprising a particular barcode, BC06 (TCTGCATCGT (SEQ ID NO: 3)). The data were analysed against a set of 8 barcodes (BC01-BC08). As demonstrated below, the query nucleic acid was aligned against (1) the full length of a reference nucleic acid comprising BC06 and then scored using the barcode of the reference and the corresponding segment of the query to provide a score of 102; (2) the correct BC06 barcode alone and then scored using the barcode to provide a score of 14; (3) the incorrect BC07 barcode alone and then scored using the barcode to provide a score of 15. Notably, the incorrect barcode BC07, which aligned to a similar location on the query as did BC06 barcode, provided a better score than the correct barcode BC06 because it was able to steal a nucleotide from the right-hand context (underlined twice).

However, when the scoring region was increased to include the barcode sequences and three contiguous nucleotides from context sequences on either side of the barcode, the correct barcode BC06 provided a score of 26 while the incorrect barcode BC07 provided a lower score of 22. This example demonstrates that the use of flanking nucleotides from the context sequence when scoring after an alignment provides discrimination of a correct barcode relative to an incorrect barcode that aligns to a similar location as the correct barcode and is capable of stealing a nucleotide from a context sequence.

```
*****Alignments to correct barcode:*****
Full sequence alignment to BC06: SEQ ID NO: 7
TGAT-GTC-ACGAGAAGAAGGGTACTTCAGTATCGTGTATTTTAAC-AGGCCGTATTAGAT
  ||| ||| |||||||||||||||||||.|.||||||||||||||| |||||||||.||||
-GATTGTCGACGAGAAGAAGGGTACTTCTGCATCGTGTATTTTAACTAGGCCGTATGAGAT
Score = 102 SEQ ID NO: 2
Barcode alignment to BC06: SEQ ID NO: 7
TGATGTCACGAGAAGAAGGGTACTTCAGTATCGTGTATTTTAACAGGCCGTATTAGAT
                                                ||.|.|||||
------------------------TCTGCATCGT------------------------
Score = 14 SEQ ID NO: 3
Flanked barcode alignment to BC06 +/− 3 bases SEQ ID NO: 7
TGATGTCACGAGAAGAAGGGTACTTCAGTATCGTGTATTTTAACAGGCCGTATTAGAT
                                          |||||.|.||||||||
---------------------ACTTCTGCATCGTGTA---------------------
Score = 26 SEQ ID NO: 4
*****Alignments to miscalled barcode (barcode sequence):*****
Barcode alignment to BC07: SEQ ID NO: 7
TGATGTCACGAGAAGAAGGGTACTTCAGTATCGTGTATTTTAACAGGCCGTATTAGAT
                                                    ||||.||||
-------------------------GAGTAGCGTG-----------------------
Score = 15 SEQ ID NO: 8
Flanked barcode alignment to BC07 +/− 3 bases: SEQ ID NO: 7
TGATGTCACGAGAAGAAGGGTACTTCAGTATCGTG-TATTTTAACAGGCCGTATTAGAT
                                          || |.||||.|||| ||
---------------------AC-TGAGTAGCGTGGTA---------------------
Score = 22 SEQ ID NO: 9
```

### Example 3

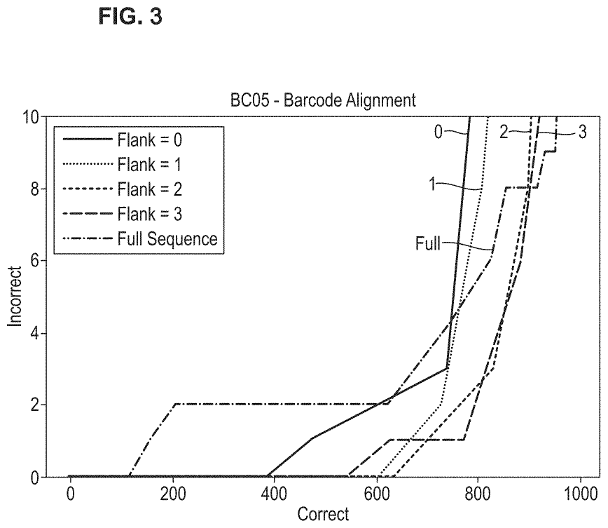

FIG. 3 provides a graph showing the number of correct and incorrect identifications of a barcoded target nucleic acid from 1000 simulated examples using reference nucleic acids comprising a fixed context sequence and a BC05 barcode. The target nucleic acid was aligned against the full length of the reference nucleic acids and then scored against either the full sequence or the barcode sequence with 0, 1, 2, or 3 flanking nucleotides. Inclusion of 1, 2 or 3 flanking bases from context sequence during the scoring phase allowed for a higher number of examples to be classified correctly before incorrect classifications were made.

### Example 4—Focused Scoring can Identify Target Nucleic Acid with High Number of Sequencing Errors

A series of simulations were performed using a query nucleic acid (e.g., target nucleic acid) comprising a particular barcode, BC06 (TCTGCATCGT (SEQ ID NO: 3)). In this example, there were 15 errors in the full sequence alignment out of 60 reference bases (25%). However, restriction of scoring to a flanked barcode (the barcode sequence and three contiguous nucleotides from context sequences on either side of the barcode) lowered the error rate such that only 2 errors were captured out of 16 reference bases (12.5%). This sequence would be classified correctly based on focused scoring, but would be discarded as incorrect based on a full scoring with a reasonable score threshold.

*****Full alignment:*****
Full sequence alignment to BC06: SEQ ID NO: 10
G-TTGACCACGAGAA-AATGG-ACTTGTGCATCGATGTATTT-AAGCTAGCACCGTAT-ATAC
| |||.|.||||||| ||.|| ||||.||||||| ||||||| || |||| .|||||| |.|
GATTGTCGACGAGAAGAAGGGTACTTCTGCATCG-TGTATTTTAA-CTAG-GCCGTATGAGAT
Score = 82 SEQ ID NO: 2
Focused scoring for BC06 +/− 3 bases:
ACTTGTGCATCGATGTA SEQ ID NO: 11
||||.||||||| ||||
ACTTGTGCATCG-TGTA SEQ ID NO: 4
Score = 28

### Example 5—Focused Scoring Prevents Misclassification Vs Full Sequence

In this example, had the flanked barcode region been considered, 4 errors would have been counted in both the correct (underlined once) and incorrect (underlined twice) barcode classifications out of 16 reference bases (25%). This sequence would not be classified with a reasonable scoring threshold. However, because there were only 4 further errors in the full context sequences, the total error rate was 13% which may exceed a reasonable scoring threshold based on full sequences.

*****Alignments to correct barcode:*****
Full sequence alignment to BC06: SEQ ID NO: 12
GATGGTCGAAGAGAAGAAGGGTACT-CTGCAACGT-TGGTTTAACTAGGTCGTATGAGAT
|||.|||||.||||||||||||||| |||||.||| |..||||||||||.||||||||||
GATTGTCGACGAGAAGAAGGGTACTTCTGCATCGTGTATTTTAACTAGGCCGTATGAGAT
Score = 96 SEQ ID NO: 2
*****Alignments to miscalled barcode (full sequence):*****
Full sequence alignment to BC04: SEQ ID NO: 12
GATGGTCGAAGAGAAGAAGGGTACTCT-GCA-AC-GT-TGGTTTAACTAGGTCGTATGAGAT
|||.|||||.||||||||||||||||| ||| || || | ||||||||||.||||||||||
GATTGTCGACGAGAAGAAGGGTACTCTCGCACACAGTAT--TTTAACTAGGCCGTATGAGAT
Score = 97 SEQ ID NO: 13

### Example 6—Scoring Against Flanked Barcodes Detection of SARS-CoV-2 Sequence

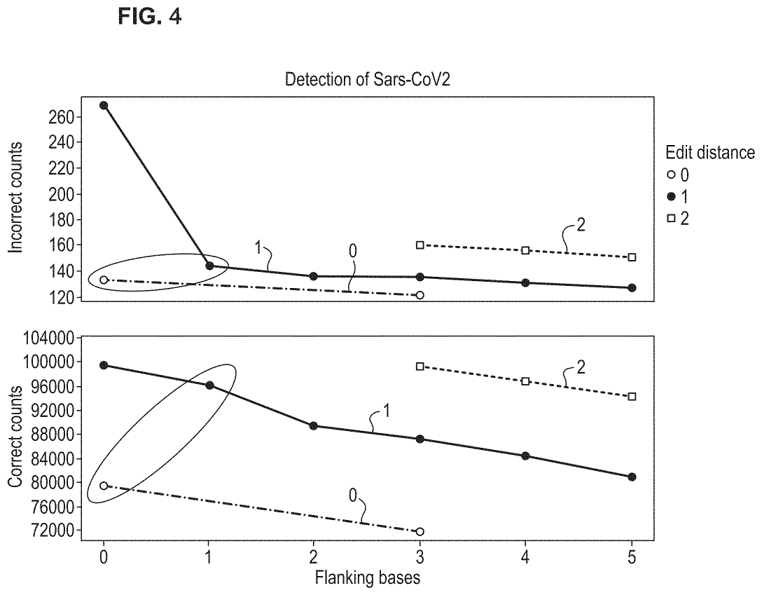

FIG. 4 provides a graph showing that the total and relative counts of incorrect and correct determinations of whether a target nucleic acid comprises a SARS-CoV-2 sequence varies depending on the number of flanking nucleotides on either side of the barcode sequence used for scoring. The number of incorrect counts can be reduced by ˜20% by increasing the number of flanking nucleotides used for scoring from 0 to 1 (with an allowed edit-distance of 1), while only reducing the number of correct counts by ˜4%. Further increases in the number of flanking nucleotides used for scoring from 1 to 5 do not continue to reduce the number of incorrect counts, however there is a further reduction in the number of correct counts (at allowed edit-distances of 0, 1 and 2)

### Example 7—Focused Scoring

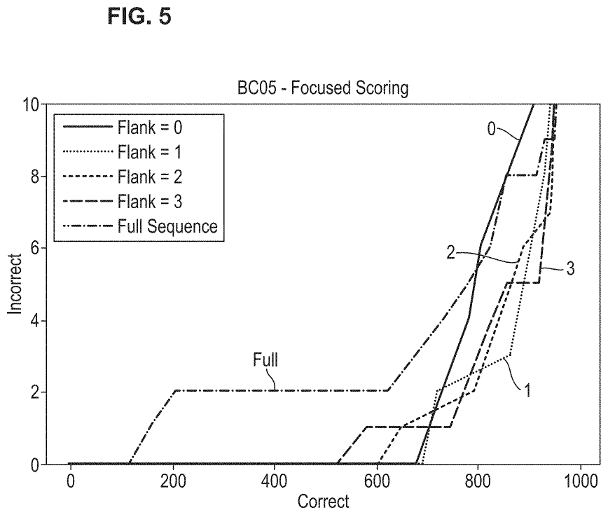

FIG. 5 provides a graph showing the number of correct and incorrect identifications of a barcoded target nucleic acid from 1000 simulated examples using reference nucleic acids comprising a fixed context sequence and a BC05 barcode. The target nucleic acid was aligned against a segment of the reference nucleic acids and then scored against either the full sequence or the barcode sequence with 0, 1, 2, or 3 flanking nucleotides.

Plotting number of correct identifications vs number of incorrect identifications from 1000 simulated examples of fixed context containing BC05. Focused scoring on the expected position of the flanked barcode sequence in the alignment achieved similar benefits to alignment of the flanked sequences. Performance was further improved relative to alignment of flanked sequences, since the focused scoring filters out spurious alignments that occur outside of the expected region (e.g. in the primer sequences). In this example, addition of 1 flanking base was sufficient to fully realise a benefit.

### Example 8

A nucleic acid sequencing read contains two barcode contexts for the AS1 target (a SARS-CoV-2 target), one of which contains the correct barcode (FIP08), the other contains a spurious alignment to an incorrect barcode (FIP02). For the correct hit (top) the addition of a flanking base to the scoring region (FL=1) had no effect on edit distance (ED), however for the incorrect hit (bottom) the addition of flanking bases increased the edit distance. In this particular case the increase in specificity of barcode assignment also led to an increase in sensitivity as this read failed QC due to conflicting barcode assignments at FL=0 but had a unique assignment at FL=1.

```
Barcode context 1 (AS1, FIP08, correct hit)
Barcode (FL = 0, ED = 1) GTGAGATGCG SEQ ID NO: 14
                                                              ||||||-|||
Barcode Context AAGCCAAAAATTTATGTGAGA-GCGCTGTGCAAAGGAATTAAGAG SEQ ID NO: 15
                                                            |||||||-||||
Barcode (FL = 1, ED = 1) TGTGAGATGCGC SEQ ID NO: 16
Barcode context 2 (AS1, FIP02, incorrect hit)
Barcode (FL = 0, ED = 1) ATGCTGCAGA SEQ ID NO: 17
                                                                            |||-||||||
Barcode Context TCAGCACACAAAACTAAAATTTATG-TGCAGA-TGTAATCTCAAGGAATTAAGGAG SEQ ID NO: 18
                                                                          ||||-||||||-
Barcode (FL = 1, ED = 2) TATGCTGCAGAC SEQ ID NO: 19
```

### Example 9—Use of Method for Multiplexed SARS-CoV-2 Testing

SARS-CoV-2 emerged in late 2019 and spread rapidly around the world, causing hundreds of thousands of COVID-19-related deaths. The discovery of the first SARS-CoV-2 genome sequence allowed the development of tests for the presence or absence of viral RNA from biological samples, which provide a way to identify people who are infected by the virus. Although there is some uncertainty about how infectious asymptomatic people are, it is more certain that many people can transmit the virus while being pre-symptomatic, or having mild symptoms. As a consequence, rather than only testing people who show symptoms, it is becoming important to enable frequent and routine screening of large numbers of people who are not presently showing symptoms, to help return to pre-pandemic activities more safely. For wide-scale screening to be worthwhile, it is important to have assays that are high throughput, accurate and very fast. Epidemiological models show that testing frequency and time-to-result are important components of a surveillance system. However, there is little benefit in being able to screen a large number of samples if the results are not made available quickly enough to inform quarantine decisions or contact tracing. In the US, many labs are taking 5-7 days or more to turn tests around.

This example describes a method which combines the rapid target-specific amplification provided by LAMP, a method of transposase-based library preparation, and real-time nanopore sequencing and data analysis. The resulting combination, LamPORE, is rapid, sensitive and highly scalable and here it demonstrated LamPORE's efficacy for detecting the presence or absence of SARS-CoV-2 RNA in clinical samples. The end-to-end procedure, beginning with 96 RNA extracts, and ending with positive and negative calls, can be performed in 115 minutes when sequencing on a MinION or GridION. The number of samples that can be sequenced in parallel can be increased by expanding either the number of LAMP barcodes or the number of ONT rapid barcodes. In these circumstances, it was useful to extend the length of the sequencing run. When using 12 different LAMP barcodes combined with 96 rapid barcodes (=1,152 samples) it was found that 4 hours of MinION sequencing was sufficient. After sequencing it is possible to remove the sample strands from the flowcell with a nuclease flush, and to load a fresh set of samples. Having a larger number of pores per flowcell, the length of the corresponding sequencing run is shorter on the PromethION, but alternatively, it was possible to use larger multiplexes.

The remaining bottleneck in the current end-to-end workflow was that of extracting RNA from the biological samples. Recent publications have indicated that saliva is a suitable source of SARS-CoV-2 RNA in infected patients, and it has been found that following heat-treatment, saliva with spiked-in inactivated SARS-Cov-2 virions can be amplified and sequenced successfully.

LAMP is capable of amplifying several targets simultaneously. LamPORE relies on sequencing, as opposed to a colour change, which raises the possibility of using a single multiplexed LamPORE reaction to detect many different pathogens. In the case of co-infection, it should also be possible to identify which combination of pathogens is present. A LamPORE assay is currently being developed to cover several viral respiratory illnesses including influenza, in multiplex.

Methods

It was shown that performing a multiplexed amplification reaction, in which three separate regions of the SARS-CoV-2 genome were targeted, performed with high sensitivity (to around Ct37 as measured by RT-qPCR). In addition, the inclusion of a fourth primer set, targeting human actin mRNA allowed true negatives to be distinguished from invalid results, where the initial sample was not taken or processed adequately. Suboptimal sampling was suspected to be responsible for false negative results in many SARS-CoV-2 tests (8, 9). Starting with RNA extracted from swabs, results were obtained from a small number of samples in approximately an hour, and from 96 samples in approximately 115 minutes. This assay was simple to scale from a small number of samples to thousands, with greater degrees of multiplexing achievable by increasing the numbers of LAMP barcodes and/or rapid barcodes.

1. Amplification and Library Preparation

Primer sequences for the amplification of three SARS-CoV-2 targets and human actin mRNA were obtained from New England Biolabs and short barcodes were added to the forward inner primers (FIP) as described. Primers were synthesised and HPLC-purified by IDT (Coralville, Iowa). The concentration of actin primers was intentionally lower than for the SARS-CoV-2 primers to prevent amplification of the human target overwhelming any SARS-CoV-2 amplification.

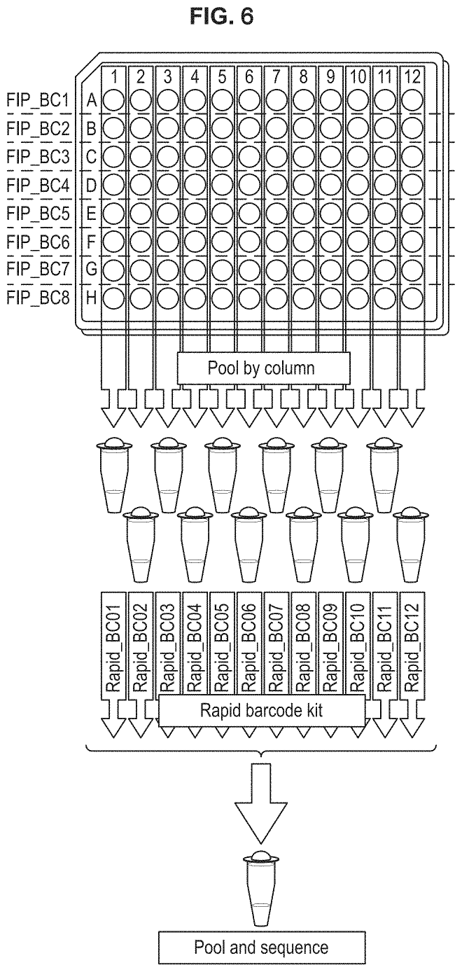

For each FIP barcode, a 10× primer pool was prepared in 400 mM guanidine hydrochloride, containing each oligonucleotide at the appropriate concentration. Reactions were performed in 96-well plates in such a way that each well in a row received the same barcoded FIP primer mix, with different barcoded FIPs being used in the different rows. Each LAMP reaction consisted of 25 μl 2× LAMP Master Mix (NEB E1700), 5 μl 10× primer pool and 20 μl RNA sample (or no-template control). Reactions were incubated at 65° C. for 35 minutes, followed by 80° C. for 5 minutes. Following amplification, reactions were pooled by column, giving 12 pools, each consisting of 8 separate reactions (FIG. 6).

Library preparation was performed separately on each of the 12 pools, in a volume of 10 μl per reaction. Each reaction consisted of 6.5 μl nuclease-free water, 1 μl of pooled LAMP product and 2.5 μl of the appropriate rapid barcode (Oxford Nanopore Technologies, SQK-RBK004). Reactions were mixed and spun down, before being incubated at 30° C. for 2 minutes and then 80° C. for 2 minutes. All reactions were then pooled into a single 1.5 ml Eppendorf LoBind tube.

The pooled products were purified using 0.8× AMPure beads, were washed in fresh 80% ethanol and were eluted in 15 μl EB buffer. 11 μl of eluate was transferred to a clean 1.5 ml Eppendorf LoBind tube, along with 1 μl rapid adapter (RAP). Reactions were incubated for 5 minutes at room temperature, before being sequenced on a single MinION flowcell for 1 hour, following the manufacturer's instructions.

2. Data Analysis

### i) Barcode and LAMP Product Identification

In order to call the presence or absence of virus in the sample, the number of reads from each LAMP target may be counted for each sample in the sequencing run. This requires the accurate identification of i) the barcode added during library preparation by the rapid barcoding kit (RBK), ii) the barcode added as part of the FIP primer during the LAMP reaction and iii) the sequence of the LAMP product associated with each target region.

The RBK barcodes are identified using the guppy_barcoder software (version 4.0.11; command line options “--barcode_kits SQK-RBK004--detect_mid_strand_barcodes--min_score_mid_barcodes 40”).

The FIP barcode was detected in a two-step process. Firstly, candidate regions were identified by aligning a sequence consisting of the FIP primer with Ns in place of the barcode sequence against all reads using the VSEARCH tool (11) (version 2.14.2; command line options: “--maxaccepts 0--maxrejects 0--id 0.75--strand both--wordlength 5--minwordmatches 2”). This returned a maximum of 2 candidate regions for each read which were subsequently filtered to remove alignments shorter than 30 nucleotides.

The second step identified the actual barcode sequence within the candidate region. A strategy was selected to maximise discrimination for these relatively short sequences. Aligning and scoring over the whole candidate region reduced discrimination due to the possibility of sequencing errors in the flanking primer regions. Restricting scoring to only the barcode sequence reduced discrimination due to alignment artifacts around the ends of the barcodes. To avoid such alignment artifacts, whilst maintaining discrimination, 1 nucleotide of the flanking primer sequence was added to each barcode before alignment within the candidate region. Each of the expanded FIP barcode sequences was aligned against the candidate region using the edlib package allowing a maximum edit distance of 1.

The LAMP product associated with each read was identified using the same VSEARCH parameters to align the genome/transcript sequence encompassed spanning the F2-B1 primer locations against each read. A valid LAMP product was detected if the alignment length is greater than 80 nucleotides and greater than 80% identity.

The multimeric nature of the LamPORE reads allowed an additional layer of quality control. Each read may only contain sequence from a single LAMP target for a single sample, therefore reads with multiple rapid barcodes, conflicting FIP barcodes or incompatible FIP-product pairings are removed from further consideration. The specific nature of the sequencing analysis allowed non-specific amplification, for example primer artefact, to be measured and excluded. Reads with RBK and FIP classifications, but which fail product classification or contain conflicting product regions, were counted as “unclassified”.

### ii) Determining Presence/Absence of SARS-CoV-2

Per-sample results of the assay were returned as either positive, negative, inconclusive, or invalid. The calls were made based on the aggregated read counts for each sample across the various targets (i.e. human actin and the three SARS-CoV-2 target regions) and cutoffs were chosen based on 1 hour of sequencing. An invalid call was returned if <50 total classified reads were obtained from across all targets (including both human actin and SARS-CoV-2). A negative call was returned if a sum of <20 reads were obtained from the three SARS-CoV-2 targets (and >=50 reads in total). An inconclusive call was returned if a sum of >=20 and <50 reads were obtained from the three SARS-CoV-2 targets. A positive call was returned if a sum of >=50 reads was obtained from the three SARS-CoV-2 targets.

iii) ROC and F1 Score Curves

To evaluate the sensitivity and specificity of the assay against the known status of 80 COVID-19 positive clinical RNA samples and a similar number of human RNA-only negatives, receiver operating characteristic (ROC) curves were generated using the metrics.roc_curve function from the scikit-learn package. The sum of read counts across each of the three SARS-CoV-2 targets (AS1, E1, and N2) served as the scoring metric for calling the results positive, negative, inconclusive, or invalid. The ROC curve therefore revealed the sensitivity and specificity of the assay at various thresholding values of that scoring metric. In addition to the curve generated for the SARS-CoV-2 read count sum, curves for read counts were also generated from each individual SARS-CoV-2 target.

The F1 score represents the harmonic mean of the sensitivity and specificity of the assay, defined as 2*[(1−FPR)*TPR]/[(1−FPR)+TPR], where TPR is the true positive rate and FPR is the false positive rate. The read count threshold (>=50 total SARS-CoV-2 target reads) was chosen in order to maximize the F1 score.

Results

### i) Assay Design

An assay that targeted a single locus from the SARS-CoV-2 genome would potentially lack robustness to sequence variants that occur as the virus evolves. To overcome this, three different regions were targeted in the SARS-CoV-2 genome, in a single multiplexed reaction. These are ORF1a and the envelope (E) and nucleocapsid (N) genes, with primer sets AS1 (10), E1 and N2 (14), respectively. In addition, as a control for the quality of the initial sample preparation, RNA extraction, reverse transcription and LAMP amplification, a set of primers were included to amplify the human actin mRNA (14). The primers target either side of a splice junction and do not amplify from genomic DNA. As long as the sample has been taken and prepared correctly, actin RNA may be present in all the swab samples, regardless of their SARS-CoV-2 status, and so this provides a way to differentiate between true negatives and invalid samples.

To assess the inclusivity of the triplex SARS-CoV-2 assay, all primer sequences to the 46,872 human SARS-CoV-2 genomes deposited at GISAID on Jun. 16, 2020 were aligned. Since not all genomes were high coverage or complete, 2,105 sequences belonging to 1,939 samples were excluded from analysis of at least one primer set because they covered fewer than 90% of all bases in that region. Of the 44,933 genomes with sufficient coverage in all three regions, 2,554 (5.68%) genomes and 179 (0.40%) genomes had a mismatch in one or two primer sets respectively, but a full match for the others. Only 2 (0.004451%) genomes had a mismatch in all three primer sets. The primer sets used have a 100% match with most sequences: 97.1% for AS1, 98.7% for E1, and 97.6% for N2. Given the widespread mutations that have been identified in SARS-CoV-2, each primer set had one mismatch for 1.3-2.9% of the strains deposited in GISAID (Table 1). The presence of a single mismatch, however, was unlikely to have a significant impact on the limit of detection, as previously shown in work on MERS-CoV LAMP assays.

TABLE 1
In silico inclusivity assay

| | AS1 | E1 | N2 |
|---|---|---|---|
| Total Primer Length (nt) | 191 | 168 | 169 |
| Total # of samples | 45,712 | 46,588 | 46,211 |
| evaluated | | | |
| 0 nt mismatches | 44,364 | 45,973 | 45,105 |
| | (97.1%) | (98.7%) | (97.6%) |
| 1 nt mismatch | 1,311 | 602 | 1,050 |
| 2 nt mismatches | 32 | 13 | 48 |
| 3 nt mismatches | 3 | 0 | 4 |
| 4+ nt mismatches | 2 | 0 | 4 |

In order to assess the potential for cross-reactivity with other viruses, the LAMP primer sequences were aligned against sequences of common viruses as well as coronaviruses related to SARS-CoV-2. Sequence identity was determined by dividing the sum of aligned primer bases by the sum of primer lengths (Table 2).

TABLE 2
Organisms assessed in silico for potential cross-reactivity
to the SARS-CoV-2

| | | AS1 | E1 | N2 |
|---|---|---|---|---|
| Pathogen | GenBank | (%) | (%) | (%) |
| Adenovirus A | NC_001460.1 | 45 | 47.6 | 46.2 |
| Adenovirus B1 | NC_011203.1 | 45.5 | 47 | 49.1 |
| Adenovirus B2 | NC_011202.1 | 44 | 44.6 | 47.9 |
| Adenovirus C | NC_001405.1 | 45 | 45.8 | 45.6 |
| Adenovirus D | NC_010956.1 | 39.8 | 47 | 49.1 |
| Adenovirus E | NC_003266.2 | 39.3 | 45.8 | 45 |
| Adenovirus F | NC_001454.1 | 41.9 | 50 | 42.6 |
| Bordetella pertussis (BPP-1) | NC_005357.1 | 38.2 | 42.9 | 46.7 |
| Candida albicans (L757) | NC_018046.1 | 43.5 | 44.6 | 43.2 |
| Chlamydia pneumoniae | NC_005043.1 | 55.5 | 58.9 | 62.7 |
| Coronavirus 229E | NC_002645.1 | 44 | 50.6 | 49.7 |
| Coronavirus HKU1 | NC_006577.2 | 43.5 | 45.8 | 46.2 |
| Coronavirus NL63 | NC_005831.2 | 45 | 50.6 | 47.3 |
| Coronavirus OC43 | NC_006213.1 | 44.5 | 47.6 | 47.3 |
| Enterovirus D68 | KP745766.1 | 39.8 | 41.7 | 44.4 |
| Haemophilus influenzae | NC_017451.1 | 60.2 | 62.5 | 65.1 |
| Human Metapneumovirus | NC_039199.1 | 40.3 | 45.2 | 43.8 |
| Influenza A (H1N1) | FJ966079.1 | 32.5 | 36.7 | 37.3 |
| Influenza A (H3N2) | KT002533.1 | 34 | 36.9 | 42.6 |
| Influenza B (Victoria) | MN230203.1 | 35.1 | 36 | 36.1 |
| Influenza B (Yamagata) | MK715533.1 | 36.6 | 37.3 | 37.3 |
| Legionella pneumophila | NZ_CP016029.2 | 62.3 | 66.1 | 66.3 |
| MERS-CoV (England 1) | NC_038294.1 | 42.9 | 48.2 | 45 |
| MERS-CoV (HCoV-EMC) | NC_019843.3 | 42.9 | 48.2 | 46.2 |
| Mycoplasma pneumoniae (C267) | NZ_CP014267.1 | 59.7 | 62.5 | 59.2 |
| Pneumocystis jirovecii | NC_020331.1 | 45.5 | 47.6 | 45.6 |
| Pseudomonas aeruginosa | NZ_CP022001.1 | 55.5 | 63.1 | 66.3 |
| Respiratory syncytial virus | NC_001803.1 | 45 | 45.2 | 46.7 |
| Rhinovirus 1 | NC_038311.1 | 38.7 | 38.7 | 44.4 |
| Rhinovirus 14 | NC_001490.1 | 39.3 | 40.5 | 38.5 |
| Rhinovirus C | NC_009996.1 | 36.6 | 41.7 | 43.8 |
| SARS-CoV-1 | NC_004718.3 | 44.5 | 96.4 | 74 |
| SARS-CoV-2 (WU) | MN908947.3 | 100 | 100 | 100 |
| Staphylococcus epidermidis | NZ_CP022247.1 | 65.4 | 65.5 | 60.9 |
| (ATCC 12228) | | | | |
| Streptococcus pneumoniae | NC_014494.1 | 61.3 | 64.9 | 65.1 |
| (AP200) | | | | |
| Streptococcus pyogenes (AP1) | NZ_CP007537.1 | 62.8 | 63.7 | 60.4 |
| Streptococcus salivarius | NC_018285.1 | 43.5 | 51.2 | 47.3 |
| (YMC-2011) | | | | |
| Human parainfluenza 1 | NC_003461.1 | 42.9 | 48.8 | 46.7 |
| Human parainfluenza 2 | NC_003443.1 | 43.5 | 41.7 | 43.8 |
| Human parainfluenza 3 | NC_001796.1 | 42.4 | 46.4 | 45 |
| Human parainfluenza 4 | NC_021928.1 | 41.9 | 48.2 | 45 |
| Tuberculosis (H37Rv) | NC_000962.3 | 53.4 | 58.9 | 61.5 |

### LamPORE Assay

SARS-CoV, which is closely related to SARS-CoV-2, was the sole virus to have a match against the total sequence length of the SARS-CoV-2 primers greater than the recommended threshold of 80%. The E-gene primer set has a match >90% with SARS-CoV, but the AS1 and N2 primer sets differ significantly, matching at only 44.5% and 74%, respectively. The likelihood of a false positive is low since SARS-CoV is not known to be in active circulation at present. Furthermore, should this situation change, the presence/absence stage of the analysis can be modified to identify positive results that are dependent entirely on amplification of the E-gene primer.

### ii) Barcode Demultiplexing

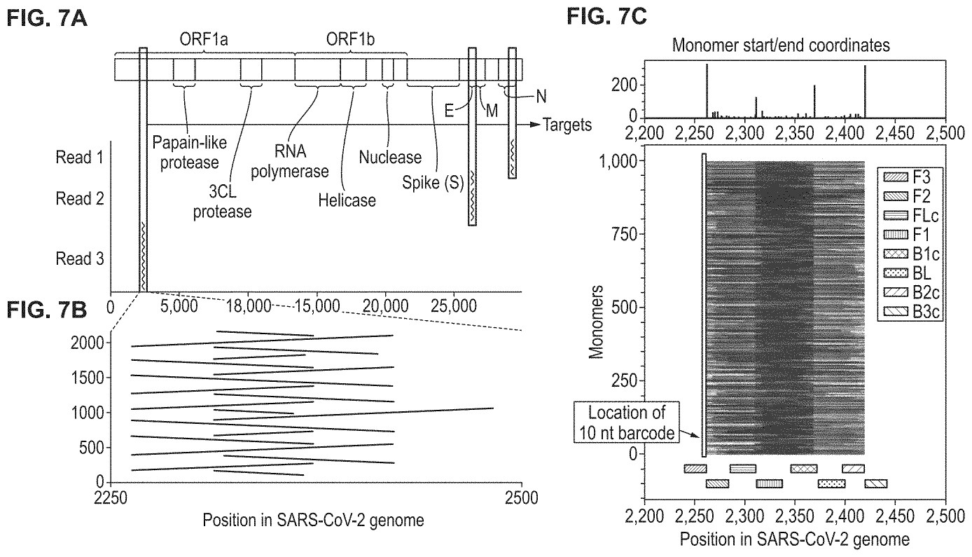

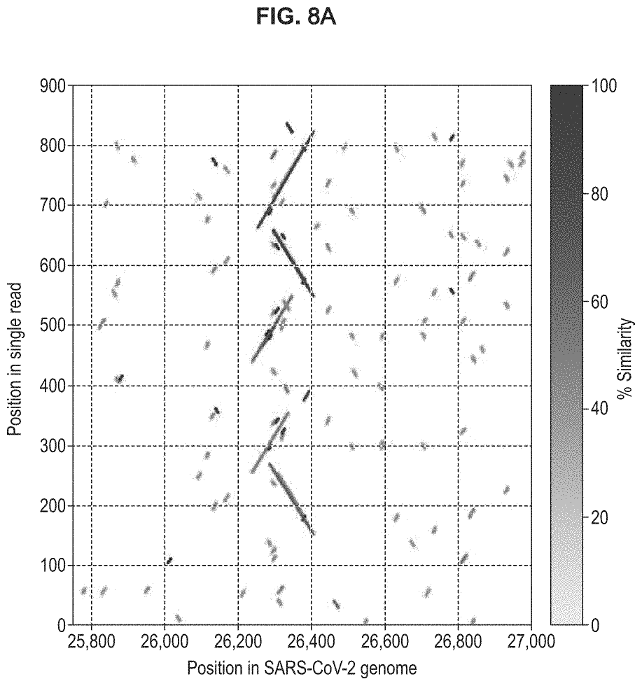

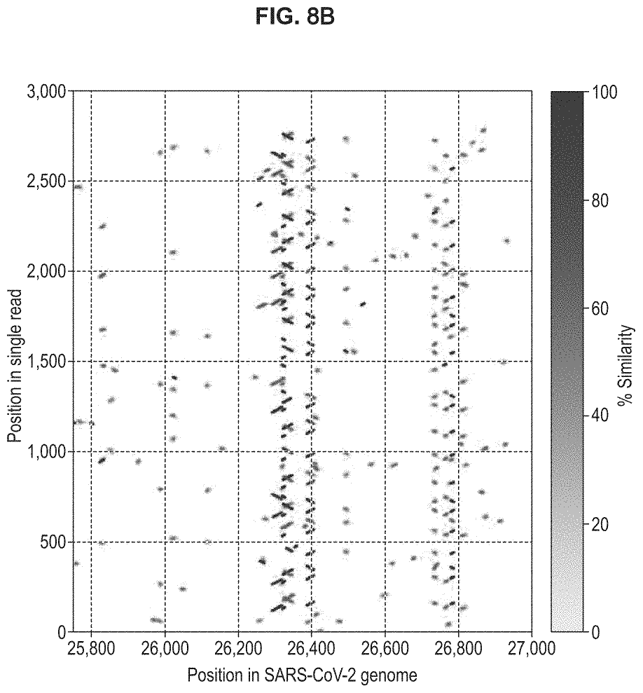

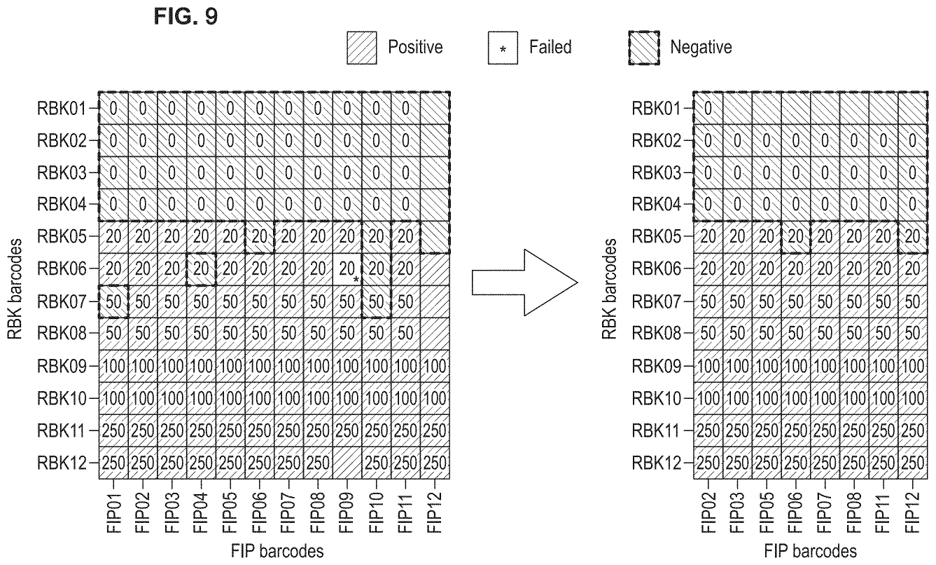

LAMP products contain multiple copies of each ˜150 bp target region joined end-to-end, forming strands of up to approximately 5 kb, with consecutive copies of the target region in alternating orientation (FIG. 7). Following library preparation with the ONT rapid kit, fragments were reduced to a modal length of around 500 bp, so still typically contain several copies of the target region.

More than one forward and reverse primer was used in each LAMP reaction at each target region, so the repeating units were not of a uniform length (FIG. 7), and because of the location of the barcodes within the FIP primer, not all copies of the repeating unit contained the LAMP barcodes. This made it possible to select reads that did contain LAMP barcodes (FIG. 7). All LamPORE reads contained an ONT barcode at one of the ends, and by selecting for LAMP barcodes, approximately 70% of reads were retained which thus contain both barcodes and the target region.

### ii) Primer Artifacts

### Generating an Alignment

Primer artifacts can accumulate during the LAMP reaction, and as a result, the consequence of judging successful amplification by a proxy measurement, such as a colour change or increase in turbidity, can be a false positive call. This is avoided when sequencing is used as a readout: reads are aligned to a reference sequence, and for a read to be considered valid, it may consist of inverted repeats of large stretches of the target region, including target-specific sequences present that do not exist in the primers. Alignments of valid reads were contiguous across the majority of the target region (FIG. 3A). In contrast, primer artifacts consist entirely of sequences covered by the primers, and these tend to align as short segments interspersed with gaps.

iii) FIP Barcode Optimisation

Verification of the FIP barcodes for each target was carried out using a dilution series of the Twist Synthetic RNA Control 2 (Twist Biosciences) for the SARS-CoV-2 loci and total human RNA extracted from GM12878 (Coriell) for the actin control. Template quantities ranged from 20-250 copies per reaction. It was observed that not only does the presence of the barcode influence the sensitivity of the reaction, the sequence of the barcode also affects performance, with some barcoded FIPs working with higher sensitivity than others. The worst-performing barcoded FIPs were excluded and in this way the initial 12 barcodes were reduced to the best-performing 8, all of which were capable of amplifying from 20 copies in a 50 μl LAMP reaction (FIG. 4). When used in combination with 12 rapid barcodes, 96 combinations are produced.

### iv) Clinical Samples

To expand the evaluation of the assay's performance, 80 clinical samples, consisting of RNA which had been extracted from nasopharyngeal swabs, were obtained. The samples had been found to be positive for SARS-CoV-2 RNA by RT-qPCR, and spanned a range of Ct values, from Ct=19 for the highest viral load to Ct=38 for the lowest. In the absence of RT-qPCR-verified negative samples, a similar number of reaction negatives using total human RNA were prepared. A sufficient number of sequences corresponding to the actin control fragment were obtained in all negative samples for these to be called as valid, and in 81 out of 85 samples, a negative call was obtained. Read-count results indicate the amplification of targets E1 or N2 in the four positives was due to contamination. Out of the 80 RT-qPCR-verified positives, 79 were called positive in the LamPORE analysis. The false negative corresponded to the lowest Ct sample, Ct=38. The two samples at Ct=37 were called positive (Table 3).

TABLE 3
Representative selection of results obtained from performing LamPORE on clinical
extracts, which had been validated as positive by RT-qPCR.

| | RT-qPCR | | | | | | | |
|---|---|---|---|---|---|---|---|---|
| Sample ID | Ct | Actin | AS1 | E1 | N2 | Unclassified | Call | True status |
| ONT5555 | 18 | 1 | 725 | 208 | 110 | 187 | POS | Positive |
| ONT1427 | 20 | 0 | 1025 | 37 | 33 | 169 | POS | Positive |
| ONT6807 | 22 | 1 | 1511 | 259 | 252 | 296 | POS | Positive |
| ONT9138 | 22 | 0 | 1634 | 302 | 285 | 325 | POS | Positive |
| ONT3768 | 22 | 2 | 1865 | 164 | 191 | 378 | POS | Positive |
| ONT9941 | 25 | 0 | 291 | 17 | 13 | 51 | POS | Positive |
| ONT2659 | 25 | 5 | 3504 | 91 | 74 | 1115 | POS | Positive |
| ONT6574 | 25 | 2 | 1229 | 61 | 54 | 306 | POS | Positive |
| ONT9410 | 26 | 1 | 1333 | 155 | 93 | 323 | POS | Positive |
| ONT0371 | 26 | 2 | 1016 | 20 | 20 | 193 | POS | Positive |
| ONT0844 | 28 | 0 | 1028 | 20 | 23 | 180 | POS | Positive |
| ONT7273 | 29 | 0 | 1257 | 1 | 3 | 163 | POS | Positive |
| ONT9661 | 29 | 1 | 550 | 0 | 7 | 92 | POS | Positive |
| ONT7343 | 30 | 3 | 1199 | 14 | 25 | 236 | POS | Positive |
| ONT2196 | 31 | 2 | 173 | 1 | 0 | 29 | POS | Positive |
| ONT7466 | 32 | 1 | 1369 | 1 | 2 | 202 | POS | Positive |
| ONT3588 | 32 | 13 | 2155 | 45 | 10 | 308 | POS | Positive |
| ONT6853 | 33 | 1 | 257 | 0 | 0 | 37 | POS | Positive |
| ONT7433 | 36 | 0 | 608 | 1 | 2 | 151 | POS | Positive |
| ONT1196 | 36 | 7 | 222 | 0 | 1 | 38 | POS | Positive |
| Human | N/A | 538 | 7 | 0 | 0 | 131 | NEG | Negative |
| RNA | | | | | | | | |
| Human | N/A | 1209 | 3 | 0 | 0 | 254 | NEG | Negative |
| RNA | | | | | | | | |
| Human | N/A | 626 | 1 | 0 | 0 | 137 | NEG | Negative |
| RNA | | | | | | | | |
| Human | N/A | 58 | 0 | 0 | 1 | 18 | NEG | Negative |
| RNA | | | | | | | | |
| Human | N/A | 211 | 4 | 0 | 0 | 49 | NEG | Negative |
| RNA | | | | | | | | |

### v) Assay Sensitivity and Specificity

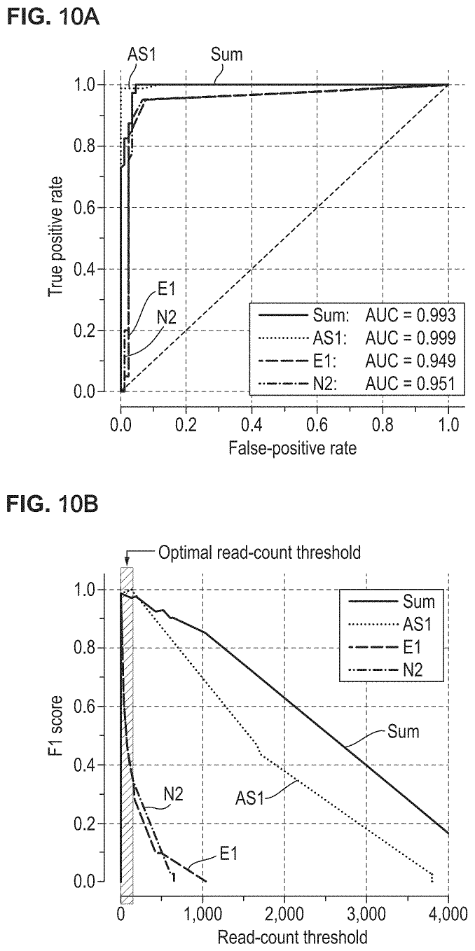

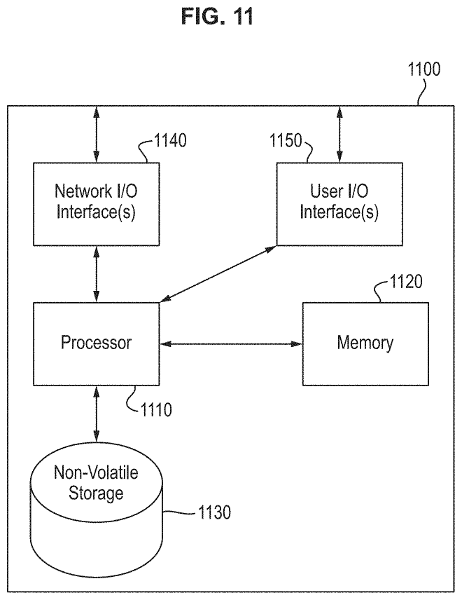

ROC curves generated from 80 COVID-19 positive clinical samples and 85 COVID-19 human RNA negatives show good concordance between SARS-CoV-2 detection via the LamPORE assay and the RT-qPCR verified status, with an area under the curve (AUC) of 0.993 for the metric used in calling the results (sum of SARS-CoV-2 target reads, FIG. 10A). 98.75% sensitivity was achieved across the 80 COVID-19 positive samples at the optimal read count threshold. Read count results suggest that contamination led to the amplification of the SARS-CoV-2 targets E1 or N2 in the four samples that generated false positive calls. The optimal read count threshold of >=50 reads for a positive call was selected by maximizing the F1 score corresponding to the ROC curves (FIG. 10B).

1. A method comprising:
using at least one computer hardware processor to perform:
(i) generating an alignment between at least a segment of a target nucleic acid and at least a segment of a reference nucleic acid, wherein the reference nucleic acid comprises a barcode sequence and a first context sequence,
(ii) determining a sequence similarity between a scoring region of the reference nucleic acid and a corresponding segment of the target nucleic acid, wherein the corresponding segment is identified based on the alignment,
wherein the scoring region comprises at least a portion of the barcode sequence and at least one and no more than a first threshold number of nucleotides of a first context sequence; and
(iii) determining whether the target nucleic acid comprises the barcode sequence based on the sequence similarity between the target nucleic acid and the scoring region of the reference nucleic acid.

2. (canceled)

3. The method of claim 1, further comprising:
prior to generating the alignment in step (i), generating an initial alignment between the at least a segment of the target nucleic acid and an initial region of the reference nucleic acid that contains at least the barcode sequence and the first context sequence,
wherein generating the alignment in step (i) is performed based on the initial alignment, and wherein the segment of the reference nucleic acid is the scoring region of the reference nucleic acid.

4. A method comprising:
using at least one computer hardware processor to perform:
(i) generating a plurality of alignments between at least a segment of a target nucleic acid and at least a segment of each of a plurality of reference nucleic acids, wherein each of the plurality of reference nucleic acids comprises a respective barcode sequence and a first context sequence;
(ii) determining a respective plurality of sequence similarities between scoring regions of the plurality of reference nucleic acids and the target nucleic acid, wherein the plurality of sequence similarities comprises a first sequence similarity, the plurality of reference nucleic acids comprises a first reference nucleic acid having a first scoring region, the plurality of respective barcode sequences comprises a first barcode sequence, and the plurality of alignments comprise a first alignment between the at least a segment of the target nucleic acid and at least a segment of the first reference nucleic acid, the determining comprising:
determining the first sequence similarity between the first scoring region of the first reference nucleic acid and a corresponding segment of the target nucleic acid, wherein the corresponding segment is identified based on the first alignment, and the first scoring region comprises at least a portion of the first barcode sequence and at least one and no more than a first threshold number of nucleotides of the first context sequence; and
(iii) identifying which of the plurality of respective barcode sequences is contained in the target nucleic acid based on the plurality of sequence similarities.

5. (canceled)

6. The method of claim 4, further comprising:
prior to generating the plurality of alignments in step (i), generating a plurality of initial alignments between the at least a segment of the target nucleic acid and an initial region of each of the reference nucleic acids that contains at least the barcode sequence and the first context sequence,
wherein generating the plurality of alignments in step (i) is performed based on the plurality of initial alignments, and wherein the segment of the first reference nucleic acid is the first scoring region of the reference nucleic acid.

7.-9. (canceled)

10. A method comprising:
using at least one computer hardware processor to perform:
(i) generating a plurality of alignments between at least a segment of each of a plurality of target nucleic acids and at least a segment of a reference nucleic acid, wherein the reference nucleic acid comprises a barcode sequence and a first context sequence;
(ii) determining a respective plurality of sequence similarities between a scoring region of the reference nucleic acid and the plurality of target nucleic acids, wherein the plurality of sequence similarities comprises a first sequence similarity, and wherein the plurality of alignments comprise a first alignment between the at least a segment of the first target nucleic acid and the reference nucleic acid, the determining comprising:
determining the first sequence similarity between the scoring region of the reference nucleic acid and a corresponding segment of the first target nucleic acid, wherein the corresponding segment is identified based on the first alignment, wherein the scoring region comprises at least a portion of the barcode sequence and at least one and no more than a first threshold number of nucleotides of the first context sequence; and
(iii) identifying which of the plurality of target nucleic acids contains the barcode sequence based on the plurality of sequence similarities.

11. (canceled)

12. The method of claim 10, further comprising:
prior to generating the plurality of alignments in step (i), generating a plurality of initial alignments between each of the plurality of target nucleic acids and an initial region of the reference nucleic acid that contains at least the barcode sequence and the first context sequence,
wherein generating the plurality of alignments in step (i) is performed based on the plurality of initial alignments, and wherein the segment of the reference nucleic acid is the scoring region of the reference nucleic acid.

13.-15. (canceled)

16. The method of claim 1, wherein the segment of the reference nucleic acid or the segment of each of the plurality of reference nucleic acids comprises the barcode sequence, at least a portion of the first context sequence, and/or at least a portion of the second context sequence.

17. The method of claim 1, wherein:
(a) the length of the segment of the reference nucleic acid or the segment of each of the plurality of reference nucleic acids is 25-50, 50-150, 100-200, 150-300, or 250-500 nucleotides; and/or
(b) the length of the barcode sequence is 4, 5, 6, 7, 8, 9, 10, 11, 12, 13, 14, 15, 15-20, or 20-25 nucleotides; and/or
(c) the length of the first context sequence is 5-10, 10-15, 15-20, 20-25, or 25-50 nucleotides; and/or
(d) the length of the second context sequence is 5-10, 10-15, 15-20, 20-25, or 25-50 nucleotides.

18.-21. (canceled)

22. The method of claim 1, wherein:
(a) the first threshold number is 1, 2, 3, 4, 5, 6, 7, 8, 9, or 10; and/or
(b) the second threshold number is 0, 1, 2, 3, 4, 5, 6, 7, 8, 9, or 10; and/or
(c) the ratio of the first threshold number relative to the length of the barcode sequence is less than or equal to 1:1, 1:2, 1:3, 1:4, 1:5, 1:6, 1:7, 1:8, 1:9, or 1:10; and/or
(d) the ratio of the second threshold number relative to the length of the barcode sequence is less than or equal to 1:1, 1:2, 1:3, 1:4, 1:5, 1:6, 1:7, 1:8, 1:9, or 1:10.

23.-25. (canceled)

26. The method of claim 1, wherein the at least one and no more than a first threshold number of nucleotides of the first context sequence in the scoring region are contiguous with the barcode sequence; and/or no more than a second threshold number of nucleotides of the second context sequence in the scoring region are contiguous with the barcode sequence.

27. (canceled)

28. The method of claim 1, wherein the scoring region comprises 1-10 nucleotides of the first context sequence and 0-10 nucleotides of the second context sequence; and/or the scoring region comprises one nucleotide of the first context sequence and one nucleotide of the second context sequence.

29. (canceled)

30. The method of claim 1, wherein generating the alignment(s) comprises generating data encoding an association between the at least a segment of the target nucleic acid and the at least a segment of the reference nucleic acid.

31. The method of claim 4, wherein generating the alignment(s) comprises generating data encoding an association between the at least a segment of the target nucleic acids and the at least a segment of each of the plurality of reference nucleic acids.

32. The method of claim 10, wherein generating the alignments comprises generating data encoding an association between the at least a segment of each of the plurality of target nucleic acids and the at least a segment of the reference nucleic acid.

33. The method of claim 1, wherein determining the sequence similarity comprises:
(a) determining a score indicative of how many nucleotides of the target nucleic acid are aligned to similar nucleotides in the scoring region of the reference nucleic acid; and/or
(b) determining a percentage of nucleotides of the target nucleic acid that are aligned to similar nucleotides in the scoring region of the reference nucleic acid; and/or
(c) determining a score indicative of how many nucleotides of the target nucleic acid are aligned to identical nucleotides in the scoring region of the reference nucleic acid; and/or
(d) determining the percentage of nucleotides of the target nucleic acid that are aligned to identical nucleotides in the scoring region of the reference nucleic acid.

34.-36. (canceled)

37. The method of claim 1, wherein the target nucleic acid or plurality of target nucleic acids is amplified prior to step (i).

38.-39. (canceled)

40. The method of claim 1, wherein the target nucleic acid or at least one of the plurality of target nucleic acids is indicative of disease or a genetic trait or marker, optionally wherein identification of the barcode sequence in the target nucleic acid indicates that a patient associated with that barcode has or previously had the disease or a genetic trait or marker, further optionally wherein the disease is a SARS-CoV-2 infection.

41.-51. (canceled)

52. A kit comprising a plurality of nucleic acids, wherein each of the plurality comprises a respective barcode having fewer than ten nucleotides and at least one fixed context sequence.

53.-56. (canceled)

57. A system, comprising:
(A) at least one computer hardware processor; and
at least one non-transitory computer readable storage medium storing processor executable instructions that, when executed by the at least one computer hardware processor, cause the at least one computer hardware processor to perform:
(i) generating an alignment between at least a segment of a target nucleic acid and at least a segment of a reference nucleic acid, wherein the reference nucleic acid comprises a barcode sequence and a first context sequence;
(ii) determining a sequence similarity between a scoring region of the reference nucleic acid and a corresponding segment of the target nucleic acid, wherein the corresponding segment is identified based on the alignment,
wherein the scoring region comprises at least a portion of the barcode sequence and at least one and no more than a first threshold number of nucleotides of a first context sequence; and
(iii) determining whether the target nucleic acid comprises the barcode sequence based on the sequence similarity between the target nucleic acid and the scoring region of the reference nucleic acid; or
(B) at least one computer hardware processor; and
at least one non-transitory computer readable storage medium storing processor executable instructions that, when executed by the at least one computer hardware processor, cause the at least one computer hardware processor to perform:
(i) generating a plurality of alignments between at least a segment of a target nucleic acid and at least a segment of each of a plurality of reference nucleic acids, wherein each of the plurality of reference nucleic acids comprises a respective barcode sequence, a first context sequence, and a second context sequence;
(ii) determining a respective plurality of sequence similarities between scoring regions of the plurality of reference nucleic acids and the target nucleic acid, wherein the plurality of sequence similarities comprises a first sequence similarity, the plurality of reference nucleic acids comprises a first reference nucleic acid having a first scoring region, the plurality of respective barcode sequences comprises a first barcode sequence, the plurality of alignments comprise a first alignment between the at least a segment of the target nucleic acid and at least a segment of the first reference nucleic acid, the determining comprising:
determining the first sequence similarity between the first scoring region of the reference nucleic acid a corresponding segment of the target nucleic acid, wherein the corresponding segment is identified based on the first alignment, wherein the first scoring region comprises at least a portion of the first barcode sequence, at least one and no more than a first threshold number of nucleotides of the first context sequence and no more than a second threshold number of nucleotides of the second context sequence; and
(iii) identifying which of the plurality of respective barcode sequences is contained in the target nucleic acid based on the plurality of sequence similarities; or
(C) at least one computer hardware processor; and
at least one non-transitory computer readable storage medium storing processor executable instructions that, when executed by the at least one computer hardware processor, cause the at least one computer hardware processor to perform:
(i) generating a plurality of alignments between at least a segment of each of a plurality of target nucleic acids and at least a segment of a reference nucleic acid, wherein the reference nucleic acid comprises a barcode sequence, a first context sequence, and a second context sequence;
(ii) determining a respective plurality of sequence similarities between a scoring region of the reference nucleic acid and the plurality of target nucleic acids, wherein the plurality of sequence similarities comprises a first sequence similarity, and wherein the plurality of alignments comprise a first alignment between the at least a segment of the first target nucleic acid and the reference nucleic acid, the determining comprising:
determining the first sequence similarity between the scoring region of the reference nucleic acid and a corresponding segment of the first target nucleic acid, wherein the corresponding segment is identified based on the first alignment, wherein the scoring region comprises at least a portion of the barcode sequence, at least one and no more than a first threshold number of nucleotides of the first context sequence and no more than a second threshold number of nucleotides of the second context sequence; and
(iii) identifying which of the plurality of target nucleic acids contains the barcode sequence based on the plurality of sequence similarities.

58. (canceled)

59. At least one non-transitory computer readable storage medium storing processor executable instructions that, when executed by at least one computer hardware processor, cause the at least one computer hardware processor to perform:
(A) (i) generating an alignment between at least a segment of a target nucleic acid and at least a segment of a reference nucleic acid, wherein the reference nucleic acid comprises a barcode sequence, a first context sequence, and a second context sequence,
(ii) determining a sequence similarity between a scoring region of the reference nucleic acid and a corresponding segment of the target nucleic acid, wherein the corresponding segment is identified based on the alignment,
wherein the scoring region comprises at least a portion of the barcode sequence, at least one and no more than a first threshold number of nucleotides of a first context sequence and no more than a second threshold number of nucleotides of a second context sequence; and
(iii) determining whether the target nucleic acid comprises the barcode sequence based on the sequence similarity between the target nucleic acid and the scoring region of the reference nucleic acid; or
(B) (i) generating a plurality of alignments between at least a segment of a target nucleic acid and at least a segment of each of a plurality of reference nucleic acids, wherein each of the plurality of reference nucleic acids comprises a respective barcode sequence, a first context sequence, and a second context sequence;
(ii) determining a respective plurality of sequence similarities between scoring regions of the plurality of reference nucleic acids and the target nucleic acid, wherein the plurality of sequence similarities comprises a first sequence similarity, the plurality of reference nucleic acids comprises a first reference nucleic acid having a first scoring region, the plurality of respective barcode sequences comprises a first barcode sequence, the plurality of alignments comprise a first alignment between the at least a segment of the target nucleic acid and at least a segment of the first reference nucleic acid, the determining comprising:
determining the first sequence similarity between the first scoring region of the reference nucleic acid a corresponding segment of the target nucleic acid, wherein the corresponding segment is identified based on the first alignment, wherein the first scoring region comprises at least a portion of the first barcode sequence, at least one and no more than a first threshold number of nucleotides of the first context sequence and no more than a second threshold number of nucleotides of the second context sequence; and
(iii) identifying which of the plurality of respective barcode sequences is contained in the target nucleic acid based on the plurality of sequence similarities; or
(C) (i) generating a plurality of alignments between at least a segment of each of a plurality of target nucleic acids and at least a segment of a reference nucleic acid, wherein the reference nucleic acid comprises a barcode sequence, a first context sequence, and a second context sequence;
(ii) determining a respective plurality of sequence similarities between a scoring region of the reference nucleic acid and the plurality of target nucleic acids, wherein the plurality of sequence similarities comprises a first sequence similarity, and wherein the plurality of alignments comprise a first alignment between the at least a segment of the first target nucleic acid and the reference nucleic acid, the determining comprising:
determining the first sequence similarity between the scoring region of the reference nucleic acid and a corresponding segment of the first target nucleic acid, wherein the corresponding segment is identified based on the first alignment, wherein the scoring region comprises at least a portion of the barcode sequence, at least one and no more than a first threshold number of nucleotides of the first context sequence and no more than a second threshold number of nucleotides of the second context sequence; and
(iii) identifying which of the plurality of target nucleic acids contains the barcode sequence based on the plurality of sequence similarities.

60.-68. (canceled)


SEQUENCE LISTING (19 sequences)

SEQ ID NO: 1 - DNA, 58 bp, Artificial Sequence
  Synthetic
gttgtcgcgagacagaagggtacacgctcgtgtagttttaactagggccgtatgagat

SEQ ID NO: 2 - DNA, 60 bp, Artificial Sequence
  Synthetic
gattgtcgacgagaagaagggtacttctgcatcgtgtattttaactaggccgtatgagat

SEQ ID NO: 3 - DNA, 10 bp, Artificial Sequence
  Synthetic
tctgcatcgt

SEQ ID NO: 4 - DNA, 16 bp, Artificial Sequence
  Synthetic
acttctgcatcgtgta

SEQ ID NO: 5 - DNA, 10 bp, Artificial Sequence
  Synthetic
ctcgcacaca

SEQ ID NO: 6 - DNA, 16 bp, Artificial Sequence
  Synthetic
actctcgcacacagta

SEQ ID NO: 7 - DNA, 58 bp, Artificial Sequence
  Synthetic
tgatgtcacgagaagaagggtacttcagtatcgtgtattttaacaggccgtattagat

SEQ ID NO: 8 - DNA, 10 bp, Artificial Sequence
  Synthetic
gagtagcgtg

SEQ ID NO: 9 - DNA, 16 bp, Artificial Sequence
  Synthetic
actgagtagcgtggta

SEQ ID NO: 10 - DNA, 58 bp, Artificial Sequence
  Synthetic
gttgaccacgagaaaatggacttgtgcatcgatgtatttaagctagcaccgtatatac

SEQ ID NO: 11 - DNA, 17 bp, Artificial Sequence
  Synthetic
acttgtgcatcgatgta

SEQ ID NO: 12 - DNA, 58 bp, Artificial Sequence
  Synthetic
gatggtcgaagagaagaagggtactctgcaacgttggtttaactaggtcgtatgagat

SEQ ID NO: 13 - DNA, 60 bp, Artificial Sequence
  Synthetic
gattgtcgacgagaagaagggtactctcgcacacagtattttaactaggccgtatgagat

SEQ ID NO: 14 - DNA, 10 bp, Artificial Sequence
  Synthetic
gtgagatgcg

SEQ ID NO: 15 - DNA, 44 bp, Artificial Sequence
  Synthetic
aagccaaaatttatgtgagagcgctgtgcaaaggaattaaggag

SEQ ID NO: 16 - DNA, 12 bp, Artificial Sequence
  Synthetic
tgtgagatgcgc

SEQ ID NO: 17 - DNA, 10 bp, Artificial Sequence
  Synthetic
atgctgcaga

SEQ ID NO: 18 - DNA, 54 bp, Artificial Sequence
  Synthetic
tcagcacacaaaactaaaatttatgtgcagatgtaatctcaaggaattaaggag

SEQ ID NO: 19 - DNA, 12 bp, Artificial Sequence
  Synthetic
tatgctgcagac